(19) United States
(12) Patent Application Publication
(10) Pub. No.: US 2025/0248379 A1
(43) Pub. Date: Aug. 7, 2025

(54) piRNA-BASED CONSTRUCTS FOR REGULATING GENE EXPRESSION AND METHODS OF USE THEREOF

(71) Applicant: Rutgers, The State University of New Jersey, New Brunswick, NJ (US)

(72) Inventors: Cory A. Henderson, Oakland, CA (US); Christina M. Bergey, New York, NY (US)

(73) Assignee: Rutgers, The State University of New Jersey, New Brunswick, NJ (US)

(21) Appl. No.: 18/855,043

(22) PCT Filed: Apr. 3, 2023
(86) PCT No.: PCT/US2023/065267

§ 371 Date: Oct. 8, 2024

Related U.S. Application Data
(60) Provisional application No. 63/362,713, filed on Apr. 8, 2022.

(51) Int. Cl. A01K 67/68; C12N 15/113; C12N 15/85

(52) U.S. Cl. CPC A01K 67/68; C12N 15/1137; C12N 15/8509; A01K 2207/05; A01K 2217/058; A01K 2227/706; C12N 2310/14; C12Y 304/17002

(57) ABSTRACT

This disclosure is based, in part, on the unexpected discovery that a piRNA-based construct is able to silence genes that are essential for transmission and/or infection of pathogens. The disclosed constructs and methods of use thereof capitalize on the role piRNAs play in transcriptional silencing and general immunity and enable development of transgenic approaches to inhibit transmission of human and crop diseases.

### CROSS-REFERENCE TO RELATED APPLICATIONS

This application claims priority under 35 U.S.C. § 119(e) to U.S. Provisional Patent Application No. 63/362,713, filed Apr. 8, 2022. The foregoing application is incorporated by reference herein in its entirety.

### FIELD OF THE INVENTION

The present invention relates generally to piRNA-based constructs for regulating gene expression and methods of use thereof.

### BACKGROUND

Transposable elements (TEs) are genetic elements that integrate into a host genome and copy into as many locations as possible. The number of integrations increases with each subsequent generation if unchecked. This process primarily occurs in the germline during oogenesis. TE insertions are harmful to host biology in many ways, most notably by disruption of normal transcription and translation of host genes. The main mechanism to prevent unchecked TE insertions is degradation of TEs while they are actively propagating via the guided cleavage activity of Argonaute (Ago) proteins by Piwi-interacting RNAs (piRNAs).

piRNAs were initially recognized for their protective role in the Drosophila germline against P-element insertions. Though essential for genome integrity, research since 2010 has suggested an additional role for piRNAs in immunity across a variety of organisms. In the canonical piRNA mediated silencing pathway, genomic piRNA clusters ultimately produce 24-30 nucleotides primary piRNAs, whose sequences match those of invasive TEs, allowing destruction of the TE that may threaten genomic stability. However, if pathogenic sequences such as retroviruses insert themselves into TE regions, piRNAs appear to be elicited against the invasive sequences during infection. Recent studies found transcripts of Anopheles-infective insect-specific viruses (ISVs) contained in Anopheles genomes, as well as potentially corresponding piRNAs, suggesting the piRNA pathway may play a role in viral immunity in Anopheles. Despite their importance in mosquito transposon control and potentially immunity, very few studies have investigated the natural variation in piRNAs or their targets in Anopheles gambiae, a major malaria vector, or other Anopheles.

### SUMMARY

This disclosure provides novel piRNA-based constructs for regulating gene expression and methods of use thereof. In one aspect, this disclosure provides a polynucleotide for regulating expression of a target gene. In some embodiments, the polynucleotide comprises a nucleotide sequence encoding one or more piRNAs or precursors thereof, wherein the one or more piRNAs binds to a Piwi or Aubergine protein and forms a RNP complex (piRC) with the Piwi or Aubergine proteins, and wherein the one or more piRNAs comprise a piRNA nucleotide sequence that hybridizes to at least a portion of a mRNA transcript of the target gene and thus causes down-regulation of transcription of the target gene.

In some embodiments, the piRNA is about 20 to about 50 nucleotides in length. In some embodiments, the piRNAs are of the same or different sequences. In some embodiments, the piRNA comprises no more than 1 in 5 base pairs of nucleotide mismatches with respect to the mRNA transcript of the target gene. In some embodiments, the piRNA has at least 90% sequence identity with the portion of the mRNA transcript of the target gene to which the piRNA hybridizes. In some embodiments, the piRNA comprises a nucleotide sequence having at least 75% sequence identity with the nucleotide sequence selected from SEQ ID NOs: 1 to 9 or comprises the nucleotide sequence selected from SEQ ID NOs: 1 to 9.

In some embodiments, the target gene is an insect-specific gene. In some embodiments, the target gene is an Anopheles gambiae-specific gene. In some embodiments, the target gene comprises An. gambiae carboxypeptidase B1 (AgCPB-1). In some embodiments, the target gene comprises a nucleotide sequence having at least 75% sequence identity with the nucleotide sequence of SEQ ID NO: 10 or comprises the nucleotide sequence of SEQ ID NO: 10.

In some embodiments, the polynucleotide comprises a nucleotide sequence encoding an An. gambiae carboxypeptidase-B2 (AgCPB-2) or variant thereof. In some embodiments, the AgCPB-2 comprises a nucleotide sequence having at least 75% sequence identity with the nucleotide sequence of SEQ ID NO: 11 or comprises the nucleotide sequence of SEQ ID NO: 11.

In some embodiments, the polynucleotide comprises a nucleotide sequence encoding an An. gambiae carboxypeptidase-B3 (AgCPB-3) or variant thereof. In some embodiments, the AgCPB-3 comprises a nucleotide sequence having at least 75% sequence identity with the nucleotide sequence of SEQ ID NO: 12 or comprises the nucleotide sequence of SEQ ID NO: 12.

In some embodiments, the one or more piRNA is located in an intronic region of the nucleotide sequence encoding AgCPB-3.

In some embodiments, the polynucleotide comprises one or more nucleotide sequences, each encoding a midgut lumen receptor blocking protein (MBP) or variant thereof. In some embodiments, the polynucleotide comprises one, two, three, four, five, or six nucleotide sequences encoding the MBP or variant thereof. In some embodiments, the MBP comprises a nucleotide sequence having at least 75% sequence identity with the nucleotide sequence of SEQ ID NO: 13 or comprises the nucleotide sequence of SEQ ID NO: 13. In some embodiments, the one or more nucleotide sequences are downstream of the nucleotide sequence encoding AgCPB-3 or variant thereof.

In some embodiments, the polynucleotide comprises an An. gambiae carboxypeptidase (AgCP) promoter operably linked to the nucleotide sequence encoding the AgCPB-3 or variant thereof. In some embodiments, the AgCP promoter comprises a nucleotide sequence having at least 75% sequence identity with the nucleotide sequence of SEQ ID NO: 14 or comprises the nucleotide sequence of SEQ ID NO: 14.

In some embodiments, the polynucleotide is configured for transformation into an An. gambiae germline. In some embodiments, the polynucleotide comprises a nucleotide sequence having at least 75% sequence identity with the nucleotide sequence of SEQ ID NO: 15 or comprises the nucleotide sequence of SEQ ID NO: 15.

In some embodiments, the nucleotide sequence encoding one or more piRNAs or precursors thereof is transcribed, and subsequently processed by Zucchini (Zuc) endoribonuclease. In some embodiments, the piRNA comprises a 5′-uracil (5′-U) residue.

In another aspect, this disclosure also provides a vector comprising the polynucleotide disclosed herein. In some embodiments, the vector is a transposon vector. In some embodiments, the vector is a piggyBac, sleeping beauty, frog prince, Tn5, Ty vector, or another modified naturally occurring transposable element.

Also within the scope of this disclosure is (a) a cell comprising a polynucleotide or a vector, as disclosed herein; (b) a composition comprising a polynucleotide, a vector, or a cell, as disclosed herein; and (c) a kit comprising a polynucleotide, a vector, a cell, or a composition, as disclosed herein.

In yet another aspect, this disclosure further provides a method for regulating the expression of a target gene in a cell. In some embodiments, the method comprises introducing into the cell a polynucleotide, a vector, a cell, or a composition, as disclosed herein, wherein the one or more piRNAs comprise a piRNA nucleotide sequence that hybridizes to at least a portion of a mRNA transcript of the target gene and thus causes down-regulation of transcription of the target gene.

In some embodiments, the cell is a somatic cell. In some embodiments, the cell is a stem cell. In some embodiments, the cell is an embryonic stem cell.

In some embodiments, the target gene is required or essential for cell growth and/or development, for mRNA degradation, for translational repression, or for transcriptional gene silencing.

In yet another aspect, this disclosure additionally provides a method of reducing transmission capability of an insect host. In some embodiments, the method comprises introducing into a cell of the insect host a polynucleotide, a vector, a cell, or a composition, as disclosed herein, wherein the one or more piRNAs comprise a piRNA nucleotide sequence that hybridizes to at least a portion of a mRNA transcript of a target gene and thus causes down-regulation of transcription of the target gene, and wherein down-regulation of the transcription of the target gene reduces transmission of a disease or an infection caused by a pathogen.

In some embodiments, the insect host comprises an An. gambiae mosquito. In some embodiments, the pathogen comprises Plasmodium falciparum. In some embodiments, the target gene is AgCPB-1. In some embodiments, the infection caused by the pathogen occurs at the midgut of the insect.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 shows a schematic diagram of an example transgenic construct designed for transformation into the An. gambiae germline. The construct comprises the An. gambiae carboxypeptidase (AgCP) promoter (the bent arrow indicates the transcription initiation site), followed by the An. gambiae carboxypeptidase-B (AgCPB-3) transcript sequence. Each of the cpb transcripts is modified in two ways: (i) by linking it to four units of a midgut lumen receptor blocking protein (MBP), and (ii) by including known An. gambiae piRNA sequences in the introns to direct piRNA biogenesis towards the included epb transcript. The construct also includes a 3×3P cyan fluorescent protein (CFP) that expresses via an eye-specific promoter for screening purposes. The arrows at the end of the construct represent the piggyBac arms.

FIG. 2 shows a photograph of fluorescent transgenic An. gambiae larvae from lines.

FIG. 3 shows relative gene expression of carboxypeptidase 1 (triangles) and 3 (circles) assayed via qPCR and using ribosomal protein S7 to standardize expression. Control samples (labeled C3, C4, and C4) are depicted on the left, while three transgenic individuals from Generation 4 (labeled G4-1, G4-2, and G4-3) from one of the two transgenic lines are included on the right. For each sample, fold change values are shown relative to the average of all wild type control samples (dashed line).

FIG. 4 shows a schematic diagram with annotations of the transgenic construct designed for transformation into the An. gambiae germline.

### DETAILED DESCRIPTION

This disclosure is based, in part, on the unexpected discovery that a piRNA-based construct is able to silence genes that are essential for transmission and/or infection of pathogens (such as vector-borne pathogens). The disclosed constructs and methods of use thereof capitalize on the role piRNAs play in transcriptional silencing and general immunity and enable development of transgenic approaches to inhibit transmission of human and crop diseases.

piRNA-Based Constructs for Regulating Gene Expression

In one aspect, this disclosure provides a polynucleotide for regulating expression of a target gene. In some embodiments, the polynucleotide comprises a nucleotide sequence encoding one or more piRNAs or precursors thereof, wherein the one or more piRNAs binds to a Piwi or Aubergine protein (e.g., Piwi or Aubergine subclass of Argonaute Proteins) and forms a RNP complex (piRC) with the Piwi or Aubergine proteins, and wherein the one or more piRNAs comprise a piRNA nucleotide sequence that hybridizes (e.g., under physiologic conditions of a cell) to at least a portion of a mRNA transcript of the target gene and thus causes down-regulation of transcription of the target gene.

In some embodiments, the coding sequence of the piRNA, when transcribed, produces the piRNA species as a transcript or a transcript that is a precursor which is metabolized by the cell to give rise to a piRNA species.

“Piwi-interacting RNA (piRNA)” is the largest class of small non-coding RNA molecules. piRNAs can be generated from long single-stranded RNA precursors that are often encoded by complex and repetitive intergenic sequences. piRNAs form RNA-protein complexes through interactions with piwi proteins. piRNA complexes have been linked to both epigenetic and post-transcriptional gene silencing of retrotransposons and other genetic elements in germ line cells, particularly those in spermatogenesis. They are distinct from microRNA (miRNA) in size, lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals, it appears that the activity of piRNAs in transposon silencing is most important during the development of the embryo, and in both C. elegans and humans, piRNAs are necessary for spermatogenesis piRNA has a role in RNA silencing via the formation of an RNA-induced silencing complex (RISC).

As used herein, the “Piwi subclass of Argonaute proteins” include mammalian as well as insect proteins that are homologs or orthologs of the Drosophila melanogaster Piwi protein. The Drosophila piwi gene was cloned and showed that it is essential for GSC maintenance in both males and females (Cox et al., Genes Dev. 12:3715-3727, 1998, incorporated herein by reference). The piwi protein is highly basic, especially in the C-terminal 100 amino acid residues, and is well conserved in evolution. Two piwi-like genes in C. elegans were also cloned, which are required for GSC renewal, and also found sequence similarity with 2 Arabidopsis thaliana proteins required for meristem cell division. By use of an EST with sequence similarity to the Drosophila piwi gene to screen a human testis cDNA library, the human homolog, PIWIL1, was further cloned. The deduced PIWIL1 protein shares 47.1% overall sequence identity, and 58.7% identity within the C-terminus, with the Drosophila protein. It was found no piwi-related genes in the bacteria and yeast genomes, suggesting that piwi has a stem cell-related function only in multicellular organisms. Piwi and piwi-related proteins differ in the N-terminus but show high homology in the C-terminus, where they all contain a conserved 43-amino acid domain, which was designated the PIWI box.

In some embodiments, examples of Piwi proteins may include Piwi, Ago3, and Aubergine (Aub).

As used herein, the “Aubergine subclass of Argonaute proteins” include mammalian as well as insect proteins that are homologs or orthologs of the Drosophila melanogaster Aubergine protein. Harris and McDonald (Development 128:2823-2832, 2001, incorporated by reference) showed that the Drosophila gene sting (Schmidt et al., Genetics 151:749-760, 1999), a member of an ancient gene family that includes the gene for the eukaryotic translation initiation factor eIF2C (Zou et al., Gene 211:187-194, 1998), is the same gene as aubergine. Four other members of the eIF2C-like gene family were also identified in the Drosophila genome. One of these is piwi (Cox et al., supra). Two additional members, CG7439 and dAGO1, are reported in the genome annotation (Adams et al., Science 287:2185-2195, 2000, incorporated by reference). The latter is the closest known relative of eIF2C in flies and is presumably the Drosophila eIF2C homolog. A fifth family member was also identified, corresponding to the genomic sequence AE003107 (Adams et al., supra) and EST clot 2083 (Rubin et al., Science 287:2222-2224, 2000, incorporated by reference), by tBLASTn searches of the BDGP databases using parts of Aub protein as the query sequence.

As used herein, “expression of a target gene” refers to the transcription and accumulation of the RNA transcript encoded by a target gene and/or translation of the mRNA into protein. The term “down-regulate” refers to any of the methods known in the art by which interfering RNA molecules reduce the level of primary RNA transcripts, mRNA or protein produced from a target gene. In some embodiments, down-regulation refers to a situation whereby the level of RNA or protein produced from a gene is reduced by at least 10%, by at least 33%, by at least 50%, or by at least 80%. In some embodiments, down-regulation refers to a reduction in the level of RNA or protein produced from a gene by at least 80%, by at least 90%, by at least 95%, or by at least 99%, e.g., within cells of the insect as compared with an appropriate control insect which has for example, not been exposed to the disclosed construct or has been exposed to a control construct. Methods for detecting reductions in RNA or protein levels are well known in the art and include RNA solution hybridization, Northern hybridization, reverse transcription (e.g., quantitative RT-PCR analysis), microarray analysis, antibody binding, enzyme-linked immunosorbent assay (ELISA), and Western blotting. In some embodiments, down-regulation refers to a reduction in RNA or protein levels sufficient to result in a detectable change in a phenotype of the insect as compared with an appropriate insect control, such as reduction of transmission and/or infection capability. Down-regulation can thus be measured by phenotypic analysis of the insect using techniques routine in the art.

In some embodiments, the piRNAs are of the same or different sequences. In some embodiments, the piRNA is about 25-39 nucleotides in length or about 26-31 nucleotides in length. In some embodiments, the minimal length of the piRNA is about 24, 25, 26, 27, 28, 29, 30, 31, 32, 33, 34, or 35 nucleotides in length. In some embodiments, the maximum length of the piRNA is no more than 100, 90, 80, 70, 60, 50, 45, 44, 43, 42, 41, 40, 39, 38, 37, 36, 35, 34, 33, 32, 31, 30, 29, 28, 27, 26, 25 nucleotides in length.

In some embodiments, the piRNA contains a nucleotide sequence that hybridizes under physiologic conditions of a cell to the nucleotide sequence of at least a portion of a genomic sequence to cause down-regulation of transcription at the genomic level, or an mRNA transcript for a gene to be inhibited (i.e., the “target” gene). The piRNA need only be sufficiently similar to natural RNA that it has the ability to mediate Piwi-dependent gene silencing. In some embodiments, the piRNA comprises no more than 1 in 5 base pairs of nucleotide mismatches with respect to the mRNA transcript of the target gene. In some embodiments, the piRNA has at least 90% (e.g., 90%, 91%, 92%, 93%, 94%, 95%, 96%, 97%, 98%, 99%, 100%) sequence identity with the portion of the mRNA transcript of the target gene to which the piRNA hybridizes.

In some embodiments, the piRNA comprises a nucleotide sequence having at least 75% (e.g., 75%, 80%, 85%, 90%, 91%, 92%, 93%, 94%, 95%, 96%, 97%, 98%, 99%, 100%) sequence identity with the nucleotide sequence selected from SEQ ID Nos: 1 to 9 or comprises the nucleotide sequence selected from SEQ ID Nos: 1 to 9.

TABLE 1
Representative sequences

| SEQ | | |
|---|---|---|
| ID | | OTHER |
| NO | NUCLEOTIDE SEQUENCE | INFORMATION |
| 1 | TACCGATGTTTCTAGCTTTACTTCGTCGTATGCTAAGACGA | piRNA-1 |
| | GG | |
| 2 | CGGGGTTTTCTATCATCTAGGCTCTTCCTGTGGCTTGTAGC | piRNA-2 |
| | TGGCTAGCCG | |
| 3 | TAGCCGCAGCTTACAAAAAGTCCGCCGAATCATATACAAG | piRNA-3 |
| | TT | |
| 4 | AATAATGGGCAAAACCGTCTCAGCCTTGAAATTCTATAGT | piRNA-4 |
| | CTCA | |
| 5 | CATTTGACAAACATCTGGTTGCCAAATGTGTCACAACTTT | piRNA-5 |
| | CACACCATCTT | |
| 6 | TAAGTGTTATTTTGTTTTGTTCTACTTGTTGC | piRNA-6 |
| 7 | GCGTTTGAAACTCCGGAGAAACTGCAACACTCAGAACCCT | piRNA-7 |
| | TTGC | |
| 8 | GGGACTCACGACGTATACTCTGTTCTTGAGCCTTCATCTCA | piRNA-8 |
| 9 | CAATGGCACATATTAATGCTCTGTAAGCCAAGGACCTGTC | piRNA-9 |
| | GTAATT | |
| 10 | ATGAGCGAAGCAATGAAGCGGCTAACATTCGTGACCGGC | AgCPB-1 |
| | TGTTTGCTGGCCTTGGCATTCGCCAAAGCCGGTTCTTATCA | coding |
| | TGAATTCGAGCTGTACAACGTGCGCCCGGAAACGGCGGA | sequence |
| | ACAGCTGTCGGTGCTGCTCAAGTGGCGCAATGGGCAGGA | |
| | GATTGAGGTGGACTTTTGGGATGCACCGAAGGTGGGCCGT | |
| | AGCGCACGCATCATGGTGACCAGGGAGGATCACAAGCGG | |
| | GTGGAAGAGTTCCTGGAGCAGCACGACATCGAGTACGAT | |
| | CTGGTGGCGGAAGATGTGCAGGAGTTGCTGAATCGAGAG | |
| | CAGCGGCGAAATGTGGAGCACGGTCGGCGGCTGAGGCGT | |
| | GACTCGAATTCGCGTGCCACTGTTAATTTCGAGCACTTCTG | |
| | GACGCTGGACGAGATCTACGAGTATCTGGACGAGCTGGC | |
| | GGTGGCGTACAATGGGCTGGTGCGCGTCTCGGAGATCGGT | |
| | CGTACGCATGAGGATCGCCCTATCAAGGCCATCACTATTT | |
| | CGACCATGGGTGCAGTCGATCAGACCCGACCGATTGTGTT | |
| | TATGGATGGAGGTATTCATGCCAGAGAATGGGCCGGCGTG | |
| | ATGTCGGTCATGTACATGATCCACGAGTTTGTGGAACATT | |
| | CGGACCAGTACGCCGAGCAGCTGTCCAACACGGACTACGT | |
| | CATCGTGCCGGTTGCCAACCCGGACGGGTACGTCTACACC | |
| | CACGAGCAGAACCGTCTGTGGCGCAAGAACCGTTCGCCG | |
| | GGCAATGTACTGTGCTACGGCGTGGACCTGAACCGCAACT | |
| | TCCCCTTCCAGTGGGATCGTACGACGAGTGAGTGTACGAA | |
| | CAACTTTGCCGGCCATGCCGCTTCCTCAGAAAACGAAACC | |
| | AAAGCACTGATCGGACTGATGGATCAGTATAAGGCCGCC | |
| | ATTCGCATGTACCTGGCGGTGCACACGTACGGCGAGATGA | |
| | TTCTGTGGCCGTGGGGTTACGATTTCCTGCACGCACCGAA | |
| | CGAGGACGATCTGCAGCGGTTGGGCGAACGGGCACGCGA | |
| | TGCACTGGTGGCGGCTGGCGGGCCCGAGTACGAGGTGGG | |
| | CAATTCGGCCGACATTCTGTACACGGCTTCCGGGGCGACG | |
| | GACGACTACGCGTACAGCCTGGGCGTGCCGTACTCGTACA | |
| | CGCTCGAGCTGACGGGCGGTGGATCGCAAGGGTTCGATCT | |
| | GCCCGCGGCCGAGCTGGCGCGCGTTACCTCGCAGACGTTC | |
| | GAGCTGCTGAAGGTGTTCGGGCAGCATGCGGGCACACTGT | |
| | CGGTGACTTCGTAA | |
| 11 | ATGCGGTTTCCAGTGCTGGTGGCGGTTTTGTGCGCGGTAA | AgCPB-2 |
| | TTGGAGGTACCGCTGGCCGCCAATCGTACAGTGGCTACAA | coding |
| | GCTCTACTCTGTGGAGCAGAACAACCAGCAGCAGGTGGA | sequence |
| | CTTCCTGCGCGAGCTGCAACAGTCCGCCCAGGATCTTGAC | |
| | TTCTGGCAGCTTGATCGTTTGGTTGGTTCCGAGGCACGGG | |
| | TGCTGGTGCCTCCGGCACAATTGGACTACTTCCGCCAGCT | |
| | GCTGAAGGCCCAGAAGTTGCGCCACCGGGAACTGATTCAC | |
| | GATTTCGAACGTGTGCAAGATGATCATCTGTACGGTACCA | |
| | AACGGGTGACTCCTTCGCAGGGTCTTCCCCTCAACAAGTA | |
| | CCTGCGGTACAACGAAATGATCGACTACATCAACACACTC | |
| | GCGAAGAAGTACTCCAGCTTTGTAACCGTTGCCGAAATTG | |
| | GCAAATCCTACGAAGGACGCCCAATCCCGGCCGTCACCAT | |
| | CCAGTCCCCGTCGCTCTACAAATCGCACCCGAACAGCTCC | |
| | AAACCGGTCGTATTCGTCGATGCTGGCATTCATGCGCGTG | |
| | AATGGGCCGCACCTGCCATGGCGATGTATCTGATCAGCGA | |
| | GCTGGTGGAGAATGCCGCCCAGCATCAGGATCTACTCGCC | |
| | GGGCTTACCTGGACGATCGTGCCAATTGCCAATCCGGACG | |
| | GGTACGAGTACAGTCACGAGCGGGAACGCCTGTGGCGCA | |
| | AAACGCGCCGTCCGGCCGGACGTAACTGTGTCGGCGTCGA | |
| | TGGCAACCGCAACTACGACTTCCACTGGGCGGAGGTCGGT | |
| | GCCTCGAATCAGCCCTGCTCCGACACATACCACGGGGAGC | |
| | AGTCGTTCTCTGAGCCGGAGACGCGCGCCATCCGGGACGA | |
| | GCTGCTGAGGTTGAAGGGACGCTGCAAGTTCTACCTGTCG | |
| | CTGCACACGTACGGACAGTACCTGCTGTACCCGTGGGGAT | |
| | GGACGTCGGAGCTGCCGGAGGACTGGCAGAAGGTCGATG | |
| | CCGTTGCGCGGGCTGGAGCACGTGCGATTGAGCAAGCGA | |
| | CCGGGTCGACCTATACGGTCGGCAGCTCCACCAATGTGCT | |
| | GTATGCGGCCGCTGGAGGCAGTGATGATTACGCGTATGCT | |
| | GTCGCCGATGTGCCGATTTCGATGACGATGGAACTGCCGG | |
| | GCGGTGGTTCGCAGGGCTTCAATCCACCGCCGACGCGCAT | |
| | CGAAGAGATCGTGAAGGAAACGTTCGTGGGCGTGCGGGC | |
| | GATGGCGTTGGAGGTGGCGCGAAACTACAGCTGA | |
| 12 | ATGAAGTACTGTGCTGCCGTGGTTGTGCTCCTAGCGCTGG | AgCPB-3 |
| | CCGTGTTTGGCCAGAGCGCCTTCGCCGAACAGGTGTCCTA | coding |
| | CCGAGACTACAAAGTCTACAGCATCCGCGTGGACACGGTG | sequence |
| | GAGAAGCACAACGTGCTGAAGCGCTGGCAGGATGTGCGC | |
| | GGGGTCGATTTCTGGGACCGTGCCGGCTACCGTGTGATGA | |
| | TCCATCCGAGCCTGCAGGAAGCGTTCGAGCGGTTCCTGAA | |
| | CCTGAACGCGTTCAGCTACGAGCGCATCATTGAGGATGTG | |
| | GAAGCCACGATCGAGGCAGAGCGCAAGTACGATCAGGAG | |
| | TACCGCCGTCGCAAGGCTGCATCCGGTCGGGCTACGGTCG | |
| | ATTTCGAGCACTTCTGGACGAATGCGGAGGTTAATGCGTA | |
| | CCTGGACGAGCTTGCCCAGACGTACCCGAATCTGGTGCGC | |
| | GTCGCCACGATTGGCACCACTCACGAGGGCCGTCCGATCA | |
| | AGTCGATCACGATCTCGACCAACAATGGTGTGGCTGGATC | |
| | GAAGCCGGTTGTCTTCATCGACGGTGGTATCCATGCACGT | |
| | GAATGGGCCGGTGTAATGTCGGTGCTCTACTTGATCCACG | |
| | AGCTGGTGGAACATTCGAGCAGCTACGCCGACATGCTGAA | |
| | CAAGGACTGGGTGATCATTCCCGTCGCCAACCCGGATGGG | |
| | TACGAGTTCTCGCACACCGACAACCGCATGTGGCGCAAGA | |
| | ACCGCTTCCCGGCCACGATCCTCTGTACCGGTATTGATCTG | |
| | AACCGTAACTGGGACTATCTGTGGGTGTTTAGCAGCAATG | |
| | CCTGCTCCGACTCGTACGCCGGAACGACCGCCTTCTCCGA | |
| | GCTGGAAACGCAAGCCCTCGATCGGGTGCTGAAGCAGTA | |
| | CGGATCGAACATTGCGGTCTATCTGGCGGTGCACACGTAC | |
| | GGCGATATGATCCTGTACCCGTACGGACACTCGTGGCCGT | |
| | TCGTGCCGGTCGCTAACCAGGCCGCCCACATTGCGCTGGG | |
| | TGAGCAGGCGCGCGATGCAATCACGGCCGTCGGTGGCCCC | |
| | CGGTACGTGGTGGGCAACAGTGCGGAAATTCTGTACACGG | |
| | CCAACGGTTGCAGCGATGATTATGTGGCCGGTGTGATCGG | |
| | CGCGCGGTACGCGTACACGCTCGAGCTGACGGGCGGTGG | |
| | CCGCAATGGGTTCGATCTGCCTGCCACGGAAATTATGGCT | |
| | GTGGCGACCCAAACCTTCCAGATCTATCGCACCATGGCGA | |
| | ACAATGCGTAA | |
| 13 | CCGTGCCAGCGCGCGATCTTCCAGTCGATCTGCAAC | MBP |
| 14 | CCGAACTCAAAACTGAAAACGCTAACACCTATTCTGAAAG | AgCP |
| | AGGGTATCCTACGTGTGGGTGGTCGTTTGCGAAACGCGCC | promoter |
| | TGTTTCGTATGAACGCAAACATCCGATAATTTTAGCGTTTT | |
| | CTCACCCATTGACCCTACTGATTGCGCGTTCTTATCATCGA | |
| | CAATATCTGCATGCGGGACAACAAGAGCTCATATCTAGCC | |
| | TGCGTGAGAGATTCTGGCCCCTTCGCGTACGCAACCTGGC | |
| | ACGAAAGGTCGTATATGAGTGTGTAAGTTGTTTCCGATCC | |
| | AAACCAACAACGGCAGAGCAAATCATGGGAGATTTGCCC | |
| | AGCGAACGCGTAAACCCAGTTCTCCCATTCTACAACACTG | |
| | GTGTGGATCTGTGTGGTCCATTATTCTATAGACAAACCAA | |
| | CAAGAAGGCTGCTCCAATCAAATGCTATGTTGCTGTTTTC | |
| | GTCTGTCTTGTGATCAAGGCAGTACATGTGGAGCTTATTG | |
| | CCGATTTGTCTACTCCAGCCTTCATTTCTACATTGAAGCGT | |
| | TTCATAGCTCGTCGCGGTAAACCATCCGTAATCCAGTGCG | |
| | ACAACGCCAAAAATTTCCGGGGAGCTGATCGAGCGCTCA | |
| | AGGAGATGTATGAGCTGTTCCAGAAACAACAACATCAGG | |
| | ATGCTGTTACAACTTACTGCGGAACGGAAGGGATCACCTT | |
| | CAACTTCATCCCTCCGCGGTCACCCCATTTAAAACGGTGG | |
| | GATCTGGGAGGCCGCAGTGAAGTCGCTGAAACGGCATCTC | |
| | AAGGCTACGATAGGATCCAGTATCTTGCGGCGAGACGACC | |
| | TGGAAACTATCTTAGTTCAAGTGGAGGCTTGTTTGAACTC | |
| | GCGACCGTTAACTGCACTTTCCAACGATCCAGAGGACCTG | |
| | GAAATTCTGACACCGGGTCATTTTTTGATTCAACGGGCGC | |
| | TTACATCGGTCCCTGAGCCATCTTATGCTGAGATTCCAGG | |
| | CAACCGCCTGGACCGCTATCAACAATTGCAGGAGTATGTT | |
| | CGGCGGATTTGGAAGCGATGGAATCGAGACTACTTGTCTG | |
| | GTTTGCATCCGCGTACCCGGTGGACTTCAAGGCGAGACAA | |
| | CGTTCGGGAGGGCACTATGGTCCTGCTGAAGGAGGACAA | |
| | CCTTCCCCCTCTCAAATGGCGCTTCGGCCGAGTGCTGAAG | |
| | ATTTATCCTGGTGATGACGGCTTGGTTCGAGTGGTTGATGT | |
| | GAAAACGAAGGATGGAATTTACAGAAGAGCCATCACCAA | |
| | GATCTGTATACTGCCCGGACAGAAGGAAGAAAGAGAGGT | |
| | TTCGTAATAAGGGTTGAAAGCTACCTTTCAACGGGGGGCG | |
| | GCTATGTTGAGTAATGAATTTGTACGGTATAAACGTATGA | |
| | CCTTCTGTCACTTGAATTTGTTGCTTGGGAATTGTATTGAG | |
| | GGGACGAATGACGGAAACAAGGAATAAAGAAAGAGTCTA | |
| | GCGCAAGCGTTCAATCAGTACAGACGTTCCTCTCTCTACC | |
| | AGTTGAGAAATTCACTAACTAAAACAGTAACAACTGAACC | |
| | GTTTTGATATCCCATCTGTTTGTATAGGTATTGATTGTGAA | |
| | CAGCAATGTGTACGATAGGAATCGTGATATGAAAATGCTG | |
| | ATCCTAAAACAGCGCTGCCTCATCGAAAGCTAGGAATCGT | |
| | TTGTGAAATGTAATAAAACGTATTTAACCTTCGTTGTAACT | |
| | ATTTTAATTGTTATCGAATTGTTTTGCTGAATCAAAACGTT | |
| | CTAAGATACATTAAAATAAACGTATGCATGGTACGATTTT | |
| | TACTTAATAACGCTTTAGCAAAATAAGGTTTGAGTCTACA | |
| | AAACTTGTTTTTACTGAATCTATTATTATTGCTTATTCACA | |
| | ATACTGATTGATGGCTTTGATAGTTCTAGAAAAATATTCA | |
| | ATTTTCGAAAACCGCATAGAAGCAGAATGGCGCAATATAT | |
| | TTTTGCTCTTGGGATTGGGGGCTGTAAGTTTGACACCTCTG | |
| | GCTTCGGTCGGATGGCGGATGGTCGTGAAGGTTCGGTTTC | |
| | TTAAGACACTATTTTCCTTATTCTTGTGAAACTAGTCTTAT | |
| | GAATTGTCACTTTTTATCTTTTTCTCTCTCTCATTCTCTCTC | |
| | TCTATTTCTTTCTCTTTCTCTCTCTCTCTTTCTCTCTCTCTCT | |
| | TTCTCTATCTCTCTCTTTCTCTATCTCGTTCTATCTCTCTAT | |
| | TTCTCACCCTATCGTTGAAAAAATCTGTTTTAGTGATGATT | |
| | GTTAAATTAAATATAAAACTTGCATTAAATATCTTCAAAC | |
| | ATTTGAAAGATAGCATTTAGAGCTACATTTCTAGCGATTA | |
| | GTTAATTAGCTAATTATTAAATTAGTAAGACGTGCACGAT | |
| | CTAATGCAAATCAACCCAGTTAACAGTGGTAAGCATGTTA | |
| | TCATAAAGAGGAGTTTATGCATTTTTATTACGCACTTCTTC | |
| | ATTCTACTTCTCTGTTCTATCAAATGAAGAAAAGTTCATTA | |
| | GAATGATCACCTCACTGTGCTGCTATCAGTATCACACCGA | |
| | AACTCTCGACCCAGTTCGTAGAATTTGCTTACCATCATTTG | |
| | CCTCATTTTTTTGCGCCTCACATCCTGAACCCGTCGTGAGC | |
| | AAGTCGCGAGCCAGACCCTGTCTGTTATTTCCATTCTCCAA | |
| | ACACCCGGTGTGGCCCATAAGTTGTACGTTTTCACACATG | |
| | TACCTGTTATCGATGACAAGCGCCATCCGTATCAGTGTGC | |
| | TGCGATCTACACCTGCATCTTGGTTCGGTTATCGCCGAGC | |
| | ATAGAATCAGCTGTTGATAGGGCAGTGCGATAAGCGTACT | |
| | TAAGAAATTGGAGCAATTATGGAGTGTTGGTTAAGGCAGG | |
| | TGGGGGAAAAGTAAATAATCACGATGGGGTACGGAATGG | |
| | GTTTGAAAACAAAAGCCAATCAAGCACATTAGCCAAAGC | |
| | ATATCTAAAGGGGGATTAATGAGAGTTTGTGTTTTGTTTA | |
| | AGCCTATTGCGCTTTCGTAAATGTTTCGTACGACAATCTCA | |
| | TGTGCTAATTCTTTGAATTTCCATGCATGAACCAAGGTTTC | |
| | CCCTTCTCAGACAGTATGCATATCTTTAGAAAGATCATAT | |
| | CGCTCTCGAACTAGATAAGATCAAGCCATTCACGATAGGC | |
| | GTGTACCAGTCAATGTTTGTCACCGTCACTCAATAAATAC | |
| | TGAGGACCTCAATACTCCAGGGTTTGGTTTACTGTTCATCC | |
| | AATTCCAGTAATCGATTCCGGTTAGTAGATTAAGCAAAGC | |
| | TGTCCACGGACAACACGCAGTCTGTGTGTTCTGCAGACAT | |
| | TCGTTATCTAATCGTACGTGATTCAATCAAACGCAATATC | |
| | GCAATGACAAGAGTTTCGCGTGCGTTCAAAGTCGTGAAAT | |
| | CATTAAGCCGCACATCATTACACACTGGGGGTTGGAAATT | |
| | TTGGCTTTTATTCGCGCGTCATAGCCGTCC | |
| 15 | CCGAACTCAAAACTGAAAACGCTAACACCTATTCTGAAAG | Entire |
| | AGGGTATCCTACGTGTGGGTGGTCGTTTGCGAAACGCGCC | sequence |
| | TGTTTCGTATGAACGCAAACATCCGATAATTTTAGCGTTTT | containing |
| | CTCACCCATTGACCCTACTGATTGCGCGTTCTTATCATCGA | above core |
| | CAATATCTGCATGCGGGACAACAAGAGCTCATATCTAGCC | elements |
| | TGCGTGAGAGATTCTGGCCCCTTCGCGTACGCAACCTGGC | |
| | ACGAAAGGTCGTATATGAGTGTGTAAGTTGTTTCCGATCC | |
| | AAACCAACAACGGCAGAGCAAATCATGGGAGATTTGCCC | |
| | AGCGAACGCGTAAACCCAGTTCTCCCATTCTACAACACTG | |
| | GTGTGGATCTGTGTGGTCCATTATTCTATAGACAAACCAA | |
| | CAAGAAGGCTGCTCCAATCAAATGCTATGTTGCTGTTTTC | |
| | GTCTGTCTTGTGATCAAGGCAGTACATGTGGAGCTTATTG | |
| | CCGATTTGTCTACTCCAGCCTTCATTTCTACATTGAAGCGT | |
| | TTCATAGCTCGTCGCGGTAAACCATCCGTAATCCAGTGCG | |
| | ACAACGCCAAAAATTTCCGGGGAGCTGATCGAGCGCTCA | |
| | AGGAGATGTATGAGCTGTTCCAGAAACAACAACATCAGG | |
| | ATGCTGTTACAACTTACTGCGGAACGGAAGGGATCACCTT | |
| | CAACTTCATCCCTCCGCGGTCACCCCATTTAAAACGGTGG | |
| | GATCTGGGAGGCCGCAGTGAAGTCGCTGAAACGGCATCTC | |
| | AAGGCTACGATAGGATCCAGTATCTTGCGGCGAGACGACC | |
| | TGGAAACTATCTTAGTTCAAGTGGAGGCTTGTTTGAACTC | |
| | GCGACCGTTAACTGCACTTTCCAACGATCCAGAGGACCTG | |
| | GAAATTCTGACACCGGGTCATTTTTTGATTCAACGGGCGC | |
| | TTACATCGGTCCCTGAGCCATCTTATGCTGAGATTCCAGG | |
| | CAACCGCCTGGACCGCTATCAACAATTGCAGGAGTATGTT | |
| | CGGCGGATTTGGAAGCGATGGAATCGAGACTACTTGTCTG | |
| | GTTTGCATCCGCGTACCCGGTGGACTTCAAGGCGAGACAA | |
| | CGTTCGGGAGGGCACTATGGTCCTGCTGAAGGAGGACAA | |
| | CCTTCCCCCTCTCAAATGGCGCTTCGGCCGAGTGCTGAAG | |
| | ATTTATCCTGGTGATGACGGCTTGGTTCGAGTGGTTGATGT | |
| | GAAAACGAAGGATGGAATTTACAGAAGAGCCATCACCAA | |
| | GATCTGTATACTGCCCGGACAGAAGGAAGAAAGAGAGGT | |
| | TTCGTAATAAGGGTTGAAAGCTACCTTTCAACGGGGGGCG | |
| | GCTATGTTGAGTAATGAATTTGTACGGTATAAACGTATGA | |
| | CCTTCTGTCACTTGAATTTGTTGCTTGGGAATTGTATTGAG | |
| | GGGACGAATGACGGAAACAAGGAATAAAGAAAGAGTCTA | |
| | GCGCAAGCGTTCAATCAGTACAGACGTTCCTCTCTCTACC | |
| | AGTTGAGAAATTCACTAACTAAAACAGTAACAACTGAACC | |
| | GTTTTGATATCCCATCTGTTTGTATAGGTATTGATTGTGAA | |
| | CAGCAATGTGTACGATAGGAATCGTGATATGAAAATGCTG | |
| | ATCCTAAAACAGCGCTGCCTCATCGAAAGCTAGGAATCGT | |
| | TTGTGAAATGTAATAAAACGTATTTAACCTTCGTTGTAACT | |
| | ATTTTAATTGTTATCGAATTGTTTTGCTGAATCAAAACGTT | |
| | CTAAGATACATTAAAATAAACGTATGCATGGTACGATTTT | |
| | TACTTAATAACGCTTTAGCAAAATAAGGTTTGAGTCTACA | |
| | AAACTTGTTTTTACTGAATCTATTATTATTGCTTATTCACA | |
| | ATACTGATTGATGGCTTTGATAGTTCTAGAAAAATATTCA | |
| | ATTTTCGAAAACCGCATAGAAGCAGAATGGCGCAATATAT | |
| | TTTTGCTCTTGGGATTGGGGGCTGTAAGTTTGACACCTCTG | |
| | GCTTCGGTCGGATGGCGGATGGTCGTGAAGGTTCGGTTTC | |
| | TTAAGACACTATTTTCCTTATTCTTGTGAAACTAGTCTTAT | |
| | GAATTGTCACTTTTTATCTTTTTCTCTCTCTCATTCTCTCTC | |
| | TCTATTTCTTTCTCTTTCTCTCTCTCTCTTTCTCTCTCTCTCT | |
| | TTCTCTATCTCTCTCTTTCTCTATCTCGTTCTATCTCTCTAT | |
| | TTCTCACCCTATCGTTGAAAAAATCTGTTTTAGTGATGATT | |
| | GTTAAATTAAATATAAAACTTGCATTAAATATCTTCAAAC | |
| | ATTTGAAAGATAGCATTTAGAGCTACATTTCTAGCGATTA | |
| | GTTAATTAGCTAATTATTAAATTAGTAAGACGTGCACGAT | |
| | CTAATGCAAATCAACCCAGTTAACAGTGGTAAGCATGTTA | |
| | TCATAAAGAGGAGTTTATGCATTTTTATTACGCACTTCTTC | |
| | ATTCTACTTCTCTGTTCTATCAAATGAAGAAAAGTTCATTA | |
| | GAATGATCACCTCACTGTGCTGCTATCAGTATCACACCGA | |
| | AACTCTCGACCCAGTTCGTAGAATTTGCTTACCATCATTTG | |
| | CCTCATTTTTTTGCGCCTCACATCCTGAACCCGTCGTGAGC | |
| | AAGTCGCGAGCCAGACCCTGTCTGTTATTTCCATTCTCCAA | |
| | ACACCCGGTGTGGCCCATAAGTTGTACGTTTTCACACATG | |
| | TACCTGTTATCGATGACAAGCGCCATCCGTATCAGTGTGC | |
| | TGCGATCTACACCTGCATCTTGGTTCGGTTATCGCCGAGC | |
| | ATAGAATCAGCTGTTGATAGGGCAGTGCGATAAGCGTACT | |
| | TAAGAAATTGGAGCAATTATGGAGTGTTGGTTAAGGCAGG | |
| | TGGGGGAAAAGTAAATAATCACGATGGGGTACGGAATGG | |
| | GTTTGAAAACAAAAGCCAATCAAGCACATTAGCCAAAGC | |
| | ATATCTAAAGGGGGATTAATGAGAGTTTGTGTTTTGTTTA | |
| | AGCCTATTGCGCTTTCGTAAATGTTTCGTACGACAATCTCA | |
| | TGTGCTAATTCTTTGAATTTCCATGCATGAACCAAGGTTTC | |
| | CCCTTCTCAGACAGTATGCATATCTTTAGAAAGATCATAT | |
| | CGCTCTCGAACTAGATAAGATCAAGCCATTCACGATAGGC | |
| | GTGTACCAGTCAATGTTTGTCACCGTCACTCAATAAATAC | |
| | TGAGGACCTCAATACTCCAGGGTTTGGTTTACTGTTCATCC | |
| | AATTCCAGTAATCGATTCCGGTTAGTAGATTAAGCAAAGC | |
| | TGTCCACGGACAACACGCAGTCTGTGTGTTCTGCAGACAT | |
| | TCGTTATCTAATCGTACGTGATTCAATCAAACGCAATATC | |
| | GCAATGACAAGAGTTTCGCGTGCGTTCAAAGTCGTGAAAT | |
| | CATTAAGCCGCACATCATTACACACTGGGGGTTGGAAATT | |
| | TTGGCTTTTATTCGCGCGTCATAGCCGTCCCGGTTTATTTT | |
| | TTATGGGATGAGATTAACTGCCTTCAGCTCGATAGCAGGA | |
| | AGAAAGGTATAAATTGTACCGCCACTGGTTAGTCGCCTTC | |
| | ATCTCTGTACGGGTCACTGTTGCAGGATTAGCTAAAAATG | |
| | AAGTACTGTGCTGCCGTGGTTGTGCTCCTAGCGCTGGCCG | |
| | TGTTTGGCCAGAGCGCCTTCGCCGAACAGGTGTCCTACCG | |
| | AGAGTGAGTGTTCTTTCACCAGACAAAGTACCGATGTTTC | |
| | TAGCTTTACTTCGTCGTATGCTAAGACGAGGCGGGGTTTT | |
| | CTATCATCTAGGCTCTTCCTGTGGCTTGTAGCTGGCTAGCC | |
| | GCAGCTTACAAAAAGTCCGCCGAATCATATACAAGTTAAT | |
| | AATGGGCAAAACCGTCTCAGCCTTGAAATTCTATAGTCTC | |
| | ACATTTGACAAACATCTGGTTGCCAAATGTGTCACAACTT | |
| | TCACACCATCTTTAAGTGTTATTTTGTTTTGTTCTACTTGTT | |
| | GCGCGTTTGAAACTCCGGAGAAACTGCAACACTCAGAACC | |
| | CTTTGCGGGACTCACGACGTATACTCTGTTCTTGAGCCTTC | |
| | ATCTCACAATGGCACATATTAATGCTCTGTAAGCCAAGGA | |
| | CCTGTCGTAATTTGTGTCCGAAAAGAGCCGAAAATTGATG | |
| | CGTGAAAAATCAATCCAATTCCAGCTACAAAGTCTACAGC | |
| | ATCCGCGTGGACACGGTGGAGAAGCACAACGTGCTGAAG | |
| | CGCTGGCAGGATGTGCGCGGGGTCGATTTCTGGGACCGTG | |
| | CCGGCTACCGTGTGATGATCCATCCGAGCCTGCAGGAAGC | |
| | GTTCGAGCGGTTCCTGAACCTGAACGCGTTCAGCTACGAG | |
| | CGCATCATTGAGGATGTGGAAGCCACGATCGAGGCAGAG | |
| | CGCAAGTACGATCAGGAGTACCGCCGTCGCAAGGCTGCAT | |
| | CCGGTCGGGCTACGGTCGATTTCGAGCACTTCTGGACGAA | |
| | TGCGGAGGTTAATGCGTACCTGGACGAGCTTGCCCAGACG | |
| | TACCCGAATCTGGTGCGCGTCGCCACGATTGGCACCACTC | |
| | ACGAGGGCCGTCCGATCAAGTCGATCACGATCTCGACCAA | |
| | CAATGGTGTGGCTGGATCGAAGCCGGTTGTCTTCATCGAC | |
| | GGTGGTATCCATGCACGTGAATGGGCCGGTGTAATGTCGG | |
| | TGCTCTACTTGATCCACGAGCTGGTGGAACATTCGAGCAG | |
| | CTACGCCGACATGCTGAACAAGGACTGGGTGATCATTCCC | |
| | GTCGCCAACCCGGATGGGTACGAGTTCTCGCACACCGACA | |
| | ACCGCATGTGGCGCAAGAACCGCTTCCCGGCCACGATCCT | |
| | CTGTACCGGTATTGATCTGAACCGTAACTGGGACTATCTG | |
| | TGGGTGTTTAGCAGCAATGCCTGCTCCGACTCGTACGCCG | |
| | GAACGACCGCCTTCTCCGAGCTGGAAACGCAAGCCCTCGA | |
| | TCGGGTGCTGAAGCAGTACGGATCGAACATTGCGGTCTAT | |
| | CTGGCGGTGCACACGTACGGCGATATGATCCTGTACCCGT | |
| | ACGGACACTCGTGGCCGTTCGTGCCGGTCGCTAACCAGGC | |
| | CGCCCACATTGCGCTGGGTGAGCAGGCGCGCGATGCAATC | |
| | ACGGCCGTCGGTGGCCCCCGGTACGTGGTGGGCAACAGTG | |
| | CGGAAATTCTGTACACGGCCAACGGTTGCAGCGATGATTA | |
| | TGTGGCCGGTGTGATCGGCGCGCGGTACGCGTACACGCTC | |
| | GAGCTGACGGGCGGTGGCCGCAATGGGTTCGATCTGCCTG | |
| | CCACGGAAATTATGGCTGTGGCGACCCAAACCTTCCAGAT | |
| | CTATCGCACCATGGCGAACAATGCGTAAGCGTTGGGGACA | |
| | ATTTTAATATAATTAAAGGTTTTTTTTTACAAAAAAAAACT | |
| | GTAACTGCATGTTTGAGATGTTCTTATTTCTTGGAAGACGG | |
| | TTACATCTCGATCAAGAGAAAGAGAGAGAGAGAAAGAGA | |
| | TGACATGCACTGCTACAGAGCTCATTAGCGTTATGGGGCT | |
| | CGCCGGGCCCGTGCCAGCGCGCGATCTTCCAGTCGATCTG | |
| | CAACGGCTCGCCGGGCCCGTGCCAGCGCGCGATCTTCCAG | |
| | TCGATCTGCAACGGCTCGCCGGGCCCGTGCCAGCGCGCGA | |
| | TCTTCCAGTCGATCTGCAACGGCTCGCCGGGCCCGTGCCA | |
| | GCGCGCGATCTTCCAGTCGATCTGCAACTAATTGTAACAG | |
| | TTAGTTATCTATTATTTAATATACTGCTATCGATACAATCG | |
| | GGCGCACGCGTGCGCCACCCAGTTCCTGATACTATCACGA | |
| | CGCCTCCCTTCCAATTAGTGAAATTGGTGATCAGGTTTTGA | |
| | AATCAACGTAAGATGTTGTCTGTGTGCAAGAACCGCATCC | |
| | AGTTTATATGGCTGTGGGTTGCGCTGGAACCGTGTCAGTC | |
| | AGACTCCTGCAACTTCCGCTGCCACAATGAAGCTACTCGC | |
| | GGTGGTGGTGTTTCTGTTCACCGTCGGCACCGTTTGCCGG | |
| | GCGGAGCAGCAAAAGTTCGAAAAGTAGGTATGCGGTACA | |
| | ATAGAAGGATTGTTCTATTTCATCAATTCACAATCCCTCCT | |
| | AACAGCTACAAGCTCTACTCACTGTACGTCGAGGATGACG | |
| | AACAGGCGGAAGAGCTACACAACTGGTACGCCGACGGGA | |
| | AGCTGGACTTCTGGAGCTACGGCACACTGAACGACAGCGT | |
| | CACCGTGATGGTGCAGCCCGATCTGCAGGAATACTTTGAG | |
| | GCTATGCTGGACGATGTGCAGCTCGAATCGGAGCTGGTAG | |
| | AGGAGAACGTACAGTCACTGCTCGAGC | |

In some embodiments, the target gene is required or essential for cell growth and/or development, for mRNA degradation, for translational repression, or for transcriptional gene silencing.

In some embodiments, the target gene comprises carboxypeptidase or variants thereof, such as carboxypeptidase A and D, depending on expression profiles in target tissues, or Fibroblast Growth Factor (FGF) if the germline were targeted or a sterile female approach were to be developed. Additional non-limiting examples of target genes include hydrolase enzymes, such as glycoside hydrolase (GH) genes. They are host factors in crop pests (capable of digesting cellulose), such as the western corn rootworm (Diabrotica virgifera virgifera), that could be used to express the genetic cargo in tissues relating to digestion to interrupt feeding on target crops. Additionally, cytosine protease (CP) and the immune gene att1 of western corn rootworm have expression patterns in the digestive tract and have been tested as targets for RNAi. They can be used as host factors that serve as target genes according to some embodiments of the invention.

In some embodiments, the target gene comprises an insect-specific gene. In some embodiments, the target gene comprises an Anopheles gambiae-specific gene. In some embodiments, the target gene comprises An. gambiae carboxypeptidase B1 (AgCPB-1). In some embodiments, the target gene comprises a nucleotide sequence having at least 75% (e.g., 75%, 80%, 85%, 90%, 91%, 92%, 93%, 94%, 95%, 96%, 97%, 98%, 99%) sequence identity with the nucleotide sequence of SEQ ID NO: 10 or comprises the nucleotide sequence of SEQ ID NO: 10.

In some embodiments, the polynucleotide comprises a nucleotide sequence encoding an An. gambiae carboxypeptidase-B2 (AgCPB-2) or variant thereof. In some embodiments, the AgCPB-3 comprises a nucleotide sequence having at least 75% (e.g., 75%, 80%, 85%, 90%, 91%, 92%, 93%, 94%, 95%, 96%, 97%, 98%, 99%) sequence identity with the nucleotide sequence of SEQ ID NO: 11 or comprises the nucleotide sequence of SEQ ID NO: 11.

In some embodiments, the polynucleotide comprises a nucleotide sequence encoding an An. gambiae carboxypeptidase-B3 (AgCPB-3) or variant thereof. In some embodiments, the AgCPB-3 comprises a nucleotide sequence having at least 75% (e.g., 75%, 80%, 85%, 90%, 91%, 92%, 93%, 94%, 95%, 96%, 97%, 98%, 99%) sequence identity with the nucleotide sequence of SEQ ID NO: 12 or comprises the nucleotide sequence of SEQ ID NO: 12.

In some embodiments, one or more piRNA is located in an intronic region of the nucleotide sequence encoding AgCPB-3.

In some embodiments, the polynucleotide comprises one or more nucleotide sequences, each encoding a midgut lumen receptor blocking protein (MBP) or variant thereof. In some embodiments, the polynucleotide comprises one, two, three, four, five, six, seven, eight, nine, or ten nucleotide sequences encoding the MBP or variant thereof. In some embodiments, the MBP comprises a nucleotide sequence having at least 75% (e.g., 75%, 80%, 85%, 90%, 91%, 92%, 93%, 94%, 95%, 96%, 97%, 98%, 99%) sequence identity with the nucleotide sequence of SEQ ID NO: 13 or comprises the nucleotide sequence of SEQ ID NO: 13. In some embodiments, one or more nucleotide sequences are downstream of the nucleotide sequence encoding AgCPB-3 or variant thereof.

In some embodiments, the polynucleotide comprises an An. gambiae carboxypeptidase (AgCP) promoter operably linked to the nucleotide sequence encoding the AgCPB-3 or variant thereof. In some embodiments, the AgCP promoter comprises a nucleotide sequence having at least 75% (e.g., 75%, 80%, 85%, 90%, 91%, 92%, 93%, 94%, 95%, 96%, 97%, 98%, 99%) sequence identity with the nucleotide sequence of SEQ ID NO: 14 or comprises the nucleotide sequence of SEQ ID NO: 14.

In some embodiments, the polynucleotide is configured for transformation into an An. gambiae germ line. In some embodiments, the polynucleotide comprises a nucleotide sequence having at least 75% (e.g., 75%, 80%, 85%, 90%, 91%, 92%, 93%, 94%, 95%, 96%, 97%, 98%, 99%) sequence identity with the nucleotide sequence of SEQ ID NO: 15 or comprises the nucleotide sequence of SEQ ID NO: 15.

In some embodiments, the nucleotide sequence encoding one or more piRNAs or precursors thereof is transcribed, and then processed by Zucchini (Zuc) endoribonuclease. In some embodiments, the piRNA comprises a 5′-U residue.

In another aspect, this disclosure also provides a vector comprising the disclosed polynucleotide. A person skilled in the art would understand any active transposable elements can be used in or as a vector for piRNA to regulate gene expression. In some embodiments, the vector can also be a naturally propagating transposon that is modified to carry and express the desired genetic cargo. In some embodiments, the vector is a transposon vector. In some embodiments, the vector is a piggyBac, sleeping beauty, frog prince, Tn5, or Ty vector.

In some embodiments, the polypeptide embodying the construct may include a transposase or an envelope protein from an active endogenous retrovirus, or a combination thereof, to enhance the performance of the gene drive component of this design. The addition of a transposase can allow for inducing mobility of the construct within the genome of the target organism, such as An. gambiae, while the envelope protein can allow for the construct to mobilize between tissue types in the target organism. Achieving one or both of these phenotypes for the disclosed construct would enhance its performance as a gene drive as they will lead to biased patterns of inheritance in which the construct is present at higher levels in each subsequent generation.

Also provided is a viral particle or virus-like particle comprising a polynucleotide or a vector, as disclosed herein.

### Cells, Compositions, and Kits

In another aspect, this disclosure provides a cell comprising a polynucleotide or a vector, as disclosed herein. In some embodiments, the cell is a somatic cell. In some embodiments, the cell is a stem cell, such as an embryonic or adult stem cell. In some embodiments, the cell is in culture or in a whole organism (i.e., in vivo).

Also within the scope of this disclosure is a method of preparing a cell or a cell culture comprising the polynucleotide or the vector, as disclosed herein. In some embodiments, the method comprises introducing into the cell the polynucleotide or the vector, as disclosed herein, and optionally culturing the cell and expanding the cell culture.

The term “culturing” or “expanding” refers to maintaining or cultivating cells under conditions in which they can proliferate and avoid senescence. For example, cells may be cultured in media optionally containing one or more growth factors, i.e., a growth factor cocktail. In some embodiments, the cell culture medium is a defined cell culture medium. The cell culture medium may include neoantigen peptides. Stable cell lines may be established to allow for the continued propagation of cells.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progenies having the same function or biological activity as screened or selected for in the originally transformed cell are included herein.

In another aspect, the polynucleotide, vector, viral particle or virus-like particle or cell, as disclosed herein, can be incorporated into compositions. The compositions generally comprise substantially isolated/purified polynucleotide, vector, viral particle or virus-like particle or cell and optionally a pharmaceutically acceptable carrier in a form suitable for being introduced into a cell. The compositions are generally formulated in full compliance with all Good Manufacturing Practice (GMP) regulations of the U.S. Food and Drug Administration. Examples of such carriers or diluents include, but are not limited to, water, saline, Ringer's solutions, and dextrose solution. The use of such media and compounds for pharmaceutically active substances is well known in the art. Except insofar as any conventional media or compound is incompatible with the disclosed composition, use thereof in the compositions is contemplated.

The disclosed polynucleotide or vector may also be admixed, encapsulated, conjugated or otherwise associated with other molecules, molecule structures or mixtures of compounds, as for example, liposomes, polymers, receptor targeted molecules, oral, rectal, topical or other formulations, for assisting in uptake, distribution and/or absorption. The polynucleotide or vector can be provided in formulations also including penetration enhancers, carrier compounds and/or transfection agents. Representative United States patents that describe the preparation of such uptake, distribution and/or absorption assisting formulations which can be adapted for delivery of RNA molecules particularly piRNA, include, but are not limited to, U.S. Pat. Nos. 5,108,921; 5,354,844; 5,416,016; 5,459,127; 5,521,291; 51543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

Also provided in this disclosure is a kit comprising a polynucleotide, a vector, a viral particle or virus-like particle, a cell, or a composition, as described above. The components of the kit may be provided in any form, e.g., liquid, dried or lyophilized form, preferably substantially pure and/or sterile. When the components of the kit are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit. In some embodiments, the kit may further include informational materials. The informational material of the kits is not limited in its form. For example, the informational material can include information about the production of the composition, concentration, date of expiration, batch or production site information, and so forth. The containers can include a unit dosage of the pharmaceutical composition. In addition to the composition, the kit can include other ingredients, such as a solvent or buffer, an adjuvant, a stabilizer, or a preservative. The kit optionally includes a device suitable for administration of the composition, e.g., a syringe or other suitable delivery device. The device can be provided pre-loaded with one or both of the agents or can be empty, but suitable for loading.

### Methods of Use

In yet another aspect, this disclosure further provides a method for regulating the expression of a target gene in a cell, such as a cell in a insect host, an animal host, or a mamal host. In some embodiments, the method comprises introducing into the cell an effective amount of a polynucleotide, a vector, a cell, or a composition, as disclosed herein, wherein the one or more piRNAs comprise a piRNA nucleotide sequence that hybridizes to at least a portion of a mRNA transcript of the target gene and thus causes down-regulation of transcription of the target gene.

With the disclosed construct, refractoriness to pathogen infection or transmission could be driven to high frequency in vector species such as Anopheles mosquitoes that transmit malaria, Aedes mosquitoes that transmit dengue viruses, or Culex mosquitoes that transmit West Nile vims. Additionally, because the piRNA pathway is common to metazoans, the method can be implemented in animals beyond arthropod vectors. As such, the disclosed construct not only can be used to reduce transmission of vector borne diseases, but can be used in any number of scenarios in which one would want to drive a desired phenotype to high frequencies in a population, such as driving insecticide susceptibility to high frequencies in crop pest populations or reducing the effects of a deleterious allele in an endangered population.

In some embodiments, the disclosed piRNA-based construct can be coupled with one or more gene drive elements, such as gene drive elements that biases inheritance, to drive the piRNA-based construct to high frequency, allowing replacement of vector (e.g., pathogen host) populations with those unable to transmit pathogens. The method can be used for other vector species and vector-borne pathogens, as well as insects such as crop pests for which driving a trait to high frequency.

In some embodiments, the cell is a somatic cell. In some embodiments, the cell is a stem cell. In some embodiments, the cell is an embryonic stem cell. In some embodiments, the cell is in culture or in a whole organism (i.e., in vivo).

In some embodiments, the target gene is required or essential for cell growth and/or development, for mRNA degradation, for translational repression, or for transcriptional gene silencing.

In some embodiments, the target gene comprises carboxypeptidase or variants thereof, such as carboxypeptidase A and D, depending on expression profiles in target tissues, or Fibroblast Growth Factor (FGF) if the germline were targeted or a sterile female approach were to be developed. Additional non-limiting examples of target genes include hydrolase enzymes, such as glycoside hydrolase (GH) genes. They are host factors in crop pests (capable of digesting cellulose), such as the western corn rootworm (Diabrotica virgifera virgifera), that could be used to express the genetic cargo in tissues relating to digestion to interrupt feeding on target crops. Additionally, cytosine protease (CP) and the immune gene att1 of western corn rootworm have expression patterns in the digestive tract and have been tested as targets for RNAi. They can be used as host factors that serve as target genes according to some embodiments of the invention.

In some embodiments, the target gene comprises an insect-specific gene. In some embodiments, the target gene comprises an Anopheles gambiae-specific gene. In some embodiments, the target gene comprises An. gambiae carboxypeptidase B1 (AgCPB-1). In some embodiments, the target gene comprises a nucleotide sequence having at least 75% (e.g., 75%, 80%, 85%, 90%, 91%, 92%, 93%, 94%, 95%, 96%, 97%, 98%, 99%) sequence identity with the nucleotide sequence of SEQ ID NO: 1 or comprises the nucleotide sequence of SEQ ID NO: 1.

In yet another aspect, this disclosure additionally provides a method of reducing transmission capability of a host (e.g., insect, animal, mammal), such as Anopheles mosquitoes that transmit malaria, Aedes mosquitoes that transmit dengue viruses, or Culex mosquitoes that transmit West Nile virus. In some embodiments, the method comprises introducing into a cell of the insect host an effective amount of a polynucleotide, a vector, a cell, or a composition, as disclosed herein, wherein the one or more piRNAs comprise a piRNA nucleotide sequence that hybridizes to at least a portion of a mRNA transcript of a target gene and thus causes down-regulation of transcription of the target gene, and wherein down-regulation of the transcription of the target gene reduces transmission of a disease or an infection caused by a pathogen.

In some embodiments, the insect host comprises an An. gambiae mosquito. In some embodiments, the pathogen comprises Plasmodium falciparum. In some embodiments, the target gene is AgCPB-1. In some embodiments, the infection caused by the pathogen occurs at the midgut of the insect.

As used herein, “effective amount” refers to the quantity or concentration of interfering RNA required to produce a phenotypic effect on a cell or an organism such that infection and/or transmission of a disease caused by the cell or the organism is reduced. In one embodiment, the phenotypic effect is reduction of infection and/or transmission of a disease, and the construct is used to achieve at least 20%, 30%, 40%, 50%, 60%, 70%, 80%, or 90% reduction of infection and/or transmission as compared to a control cell or organism. In some embodiments, the disclosed construct or method can be used to achieve at least 20%, 30%, 40%, 50%, 60%, 70%, 80%, or 90% reduction of infection and/or transmission of a disease.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “polynucleotide” refers to a single or double stranded nucleic acid sequence which is provided in the form of a DNA or RNA sequence, a complementary polynucleotide sequence (cDNA), a genomic polynucleotide sequence, and/or a composite polynucleotide sequences (e.g., a combination of the above).

“Nucleic acid,” “oligonucleotide,” or “polynucleotide” as used herein refers to at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single-stranded or double-stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine, and isoguanine. Nucleic acids may be obtained by chemical synthesis methods or by recombinant methods.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules.

“Hybridization” means the Watson-Crick base-pairing of essentially complementary nucleotide sequences (polymers of nucleic acids) to form a double-stranded molecule.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide; for example, unique subsequences (e.g., as determined by standard sequence comparison and alignment techniques) of the polynucleotides disclosed herein are also encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide, or peptide. Functional variants may be naturally occurring or may be man-made.

Variants may be (i) one in which one or more of the amino acid residues are substituted with a conserved or non-conserved amino acid residue (e.g., a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The percent identity between two amino acid sequences can be determined using the algorithm of E. Meyers and W. Miller (Comput. Appl. Biosci., 4:11-17 (1988)), which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm, which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of this disclosure. To obtain gapped alignments for comparison purposes, Gapped BLAST can be utilized as described in Altschul et al. (1997) Nucleic Acids Res. 25 (17): 3389-3402. When utilizing BLAST and Gapped BLAST programs, the default parameters of the respective programs (e.g., XBLAST and NBLAST) can be used. (See www.ncbi.nlm.nih.gov).

“Regulated promoter” refers to promoters that direct gene expression not constitutively, but in a temporally- and/or spatially-regulated manner, and includes both tissue-specific and inducible promoters. It includes natural and synthetic sequences as well as sequences which may be a combination of synthetic and natural sequences. Different promoters may direct the expression of a gene in different tissues or cell types, or at different stages of development, or in response to different environmental conditions. New promoters of various types useful in plant cells are constantly being discovered, numerous examples may be found in the compilation by Okamuro et al. (1989).

“Inducible promoter” refers to those regulated promoters that can be turned on in one or more cell types by an external stimulus, such as a chemical, light, hormone, stress, or a pathogen.

“Constitutive expression” refers to expression using a constitutive or regulated promoter. “Conditional” and “regulated expression” refer to expression controlled by a regulated promoter.

“Constitutive promoter” refers to a promoter that is able to express the open reading frame (ORF) that permits constitutive expression.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation.

As used herein, the term “recombinant” refers to a cell, microorganism, nucleic acid molecule or vector that has been modified by the introduction of an exogenous nucleic acid molecule or has controlled expression of an endogenous nucleic acid molecule or gene. Deregulated or altered to be constitutively altered, such alterations or modifications can be introduced by genetic engineering. Genetic alteration includes, for example, modification by introducing a nucleic acid molecule encoding one or more proteins or enzymes (which may include an expression control element such as a promoter), or addition, deletion, substitution of another nucleic acid molecule, or other functional disruption of, or functional addition to, the genetic material of the cell. Exemplary modifications include modifications in the coding region of a heterologous or homologous polypeptide derived from the reference or parent molecule or a functional fragment thereof.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent, or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes, oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

Vector-borne diseases are a leading cause of morbidity and mortality in sub-Saharan Africa, the most notable example being malaria, transmitted by mosquitoes of the genus Anopheles and responsible for 30-50% of outpatient visits to Ugandan health facilities. Because pathogens must first evade the mosquito immune system to be transmitted to humans, transgenic manipulation of the mosquito immune response is a potential control strategy. Here, piwi interacting RNAs (piRNAs), an understudied but important component of the mosquito immune response to pathogens, was investigated using natural and laboratory populations. To this end, transgenic Anopheles were generated to study this potential targeted piRNA-mediated silencing, with the ultimate goal of producing a mosquito refractory to malaria.

piRNAs were initially recognized for their protective role in the Drosophila germline against P-element insertions. Though essential for genome integrity, research since 2010 has suggested an additional role for piRNAs in immunity across a variety of organisms. In the canonical piRNA mediated silencing pathway, genomic piRNA clusters ultimately produce 24-30 nucleotides primary piRNAs, whose sequences match those of invasive transposable elements (TEs), allowing destruction of the TE that may threaten genomic stability. However, if pathogenic sequences such as retroviruses insert themselves into TE regions, piRNAs appear to be elicited against the invasive sequences during infection. Recent studies found transcripts of Anopheles-infective insect specific viruses (ISVs) contained in Anopheles genomes, as well as potentially corresponding piRNAs, suggesting the piRNA pathway may play a role in viral immunity in Anopheles.

Despite their importance in mosquito transposon control and potentially immunity, very few studies have investigated the natural variation in piRNAs or their targets in An. gambiae, a major malaria vector, or other Anopheles. Understanding piRNA evolution is essential for transgenic methods of pathogen control. If mosquitoes were to evolve piRNAs targeting a transgenic construct, the inhibition may halt the spread of the construct via gene drive. Conversely, however, the potential importance of piRNAs to Anopheles immunity suggests an exciting novel pathogen control mechanism—by exposing Anopheles mosquitoes to modified TEs carrying pathogenic transcripts or host transcripts necessary for pathogenic replication, a piRNA response to the targeted transcripts could be elicited, thereby establishing immunity to the pathogen in the mosquito vector.

piRNA-Mediated Immunity

Mosquitoes are under constant pressure from numerous bloodborne microbiota that may be infectious to the vector itself and/or to the organisms, such as humans, on which the vector feeds. Because of their consistent exposure to potential pathogens, vector mosquitoes employ both physical and physiological barriers that resist the entry and subsequent establishment of pathogens within the mosquito. Primarily these responses take place in the midgut of the mosquito. However, if infection is established, the insect mounts a vigorous innate cellular and humoral immune response against the pathogen through three major mechanisms: phagocytosis, melanization, and lysis. During these processes, various signaling pathways are activated, including classical pathways such as immune deficiency, JAK/STAT, and Leu-rich repeat immune factors, as well as RNA interference (RNAi), metabolism, energy production, and transport.

In addition to genomic stability functions, studies of Aedes mosquitoes and An. coluzzii have demonstrated that piRNA clusters are coded within host genomes with targets that are not expressly TEs, but which instead have important immune function roles, including host transcripts and viral elements. The hypothesized importance of piRNAs in Anopheles immunity is supported by previous studies, which have found large segments of insect-specific virus (ISV) transcripts integrated into the genome of natural An. coluzzii individuals. In one study, upon infection of these mosquitoes with O'nyong'nyong virus, piRNAs were elicited that had sequence complementarity to O'nyong'nyong virus transcripts. Also, recent work on koalas has demonstrated that a retroviral insert in an existing TE in the koala genome led to piRNA production against the retroviral transcript and increased resistance to the virus, while in populations where the retroviral transcript insertion occurred outside of a TE insertion, no alterations to immunity or piRNA response were observed (Yu et al., 2019).

### Host Factor Target Choice

As Plasmodium does not have a stage of development in which the transcripts it produces are present within the cytoplasm of the mosquito host cells, the host factor target for maintaining the TE in the population should be a mosquito host factor whose presence is necessary for one of the developmental life stages of P. falciparum as well as one that reduced fertility of the mosquito when silenced. Carboxypeptidase B1 (CPB1) in An. gambiae, a protease that preferentially acts upon basic amino acids such as arginine and lysine, has been shown to be up-regulated by infection of Anopheles mosquitoes with Plasmodium parasites. Further, antigens against CPB1 in Anopheles and transgenic mosquitoes with non-effective CBP1 have demonstrated a marked reduction in oocyst formation and sporogony, greatly reducing reproductive capacity alongside the simultaneous reduction in malaria transmission potential. Another carboxypeptidase variant in An. gambiae (CPB2) has also demonstrated potential links to fertility and upregulation in response to Plasmodium infection, but it has not been demonstrated that reducing expression of this host factor leads to a reduction in Plasmodium infection or transmission rates.

There are two outcomes that were attempted to avoid through the transgenic construct design. First, initial increased expression of the included host factor was expected since additional copies of the host factor were introduced. As a result, there is a risk of actually generating mosquitoes with increased fertility, at least prior to piRNA cluster formation. Second, piRNA cluster formation could arise and silence the transgene, essentially negating the transgenic modification. To mitigate both of these risks, a copy of cpb/in the genome was chosen that is similar to the one related to fertility but is not itself important for fertility (cpb3). The included genetic cargo that will block malaria transmission is a linker on the transgenic cpb3 insert. It will block receptors necessary for Plasmodium infection of the midgut. The addition of the receptor blocking peptide will block necessary receptors in the interior of the midgut lumen for ookinete to oocyst transformation. Under normal non-silenced scenarios, increased expression of the host factor CPB3 and the receptor blocker are expected, and in a scenario where the integrated TE sequences form piRNA clusters, the related fertility host factor CPB1 should be silenced as well, resulting in reduced fertility and transmission capability. Essentially, resistance against the transgenic construct would also cause silencing of a host factor necessary for fertility. Silencing of cpb1 due to piRNA targeting of the integrated TE sequences should both prevent the silencing from being represented in the next generation due to the fertility impacts while still preventing malaria transmission in the living female. Once the TE has made it to population fixation, more and more instances of piRNA-mediated repression of epb genes will occur, impacting vector population numbers, but its presence at ubiquitous levels will prevent its removal due to purifying selection.

Thus, this system provides three separate avenues for blocking malaria transmission depending on the stage of TE integration and piRNA production in the mosquito population: i) via blockage of midgut lumen receptors required for malaria oocyst formation, ii) via piRNA mediated down-regulation of the epb genes required for the midgut stage of malaria infection in the vector mosquito once piRNA expression is initiated, and iii) via repression of vector populations due to reduced fecundity in response to cpb down-regulation once TE integrations and piRNA cluster formation is ubiquitous.

### Design and Injection of Modified Transposable Element

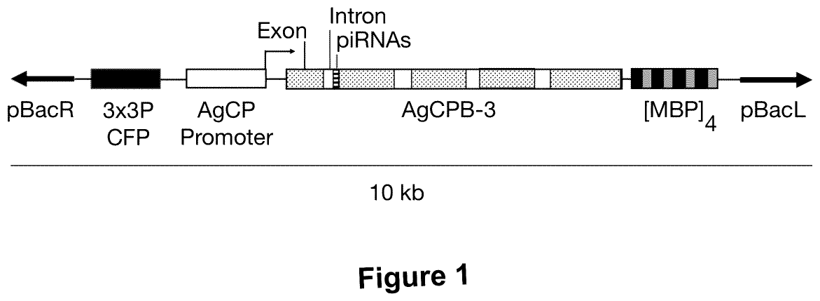

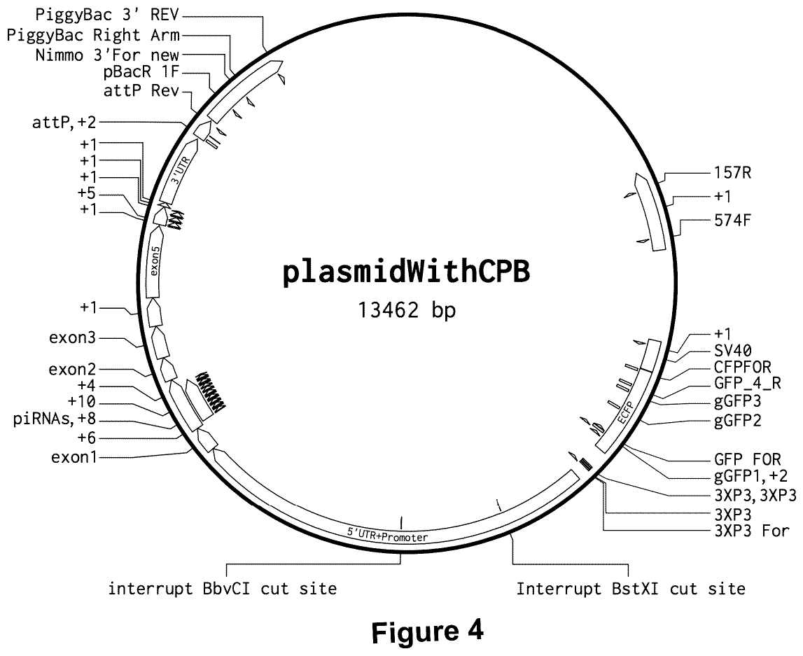

A transgenic approach was established to determine if a piRNA response can be elicited against a targeted transcript. Specifically, TEs were designed using a piggyBac vector as the backbone, a fluorescent marker that expresses in the eyes for screening purposes, a promoter for the expression of the host factor after ingestion of a bloodmeal, and the host transcript linked to the receptor blocking peptide required for Plasmodium invasion of the midgut. In addition, known piRNA sequences from An. gambiae were included in intronic regions near the beginning of the first exon to direct piRNA biogenesis towards the included cpb3 transcript. A schematic of the construct is provided (FIG. 1), and a schematic of the plasmid with annotations is also provided (FIG. 4).

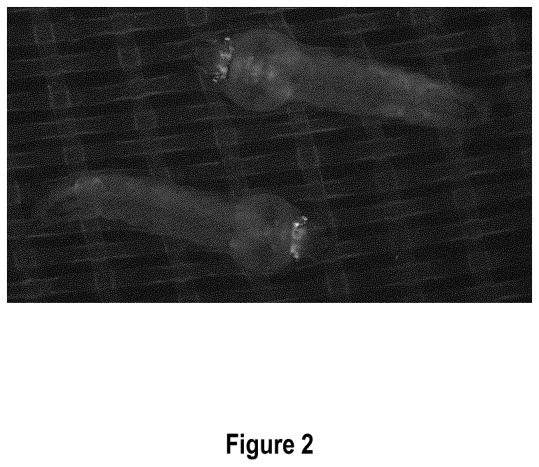

Two replicate transgenic An. gambiae lines were generated via embryo injection protocol and maintained at the University of Maryland's Insect Transformation Facility (FIG. 2).

piRNA Cluster Formation and Gene Silencing Assay

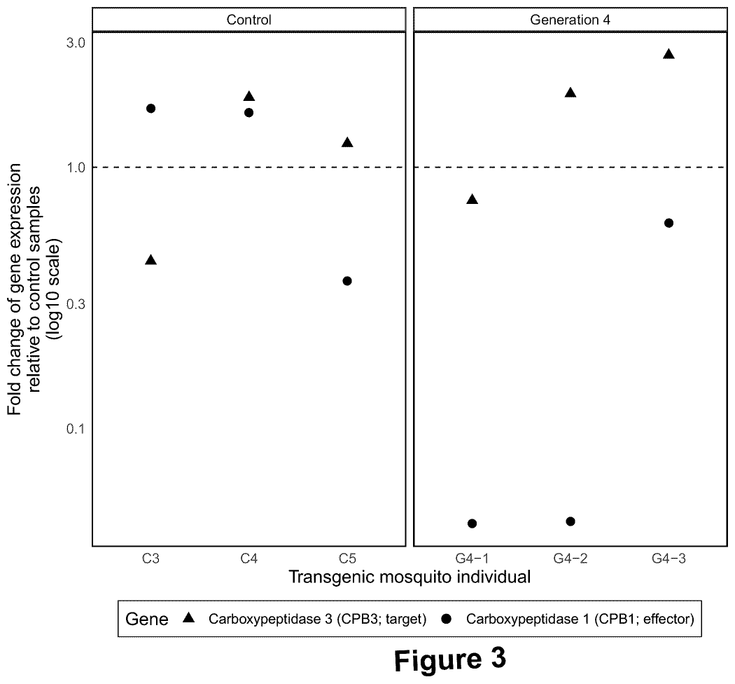

As initial generations following TE insertion will carry the TEs but likely not yet have had the TEs converted into a piRNA cluster, the expression of the included cpb3 gene and its related copies were tracked via qPCR. DNA and RNA were extracted from whole carcasses that were macerated with disposable plastic pestles using the Qiagen AllPrep DNA/RNA Mini kit. qPCR was performed with SYBR green kits using primers published previously for cpb1 and newly developed primers for cpb3 (Lavazec et al., Infect Immun. 2007 April; 75 (4): 1635-42 (2007)). Fold change of cpb1 and cpb3 compared to a housekeeping gene, ribosomal protein S7, is calculated using the delta-delta CT method with 40 qPCR cycles and an annealing temp of 55° C. Here, data from three individuals with three replicates are provided, each from G4 of one of the two transgenic lines, which demonstrate silencing of cpb1/3 compared with wild type control in a subset of samples (FIG. 3). Notably, although cpb3 expression in the transgenic lines in the absence of silencing should have elevated expression due to the increased copy number of the gene in the modified transgenic individuals, expression levels comparable to wild type mosquitoes was observed despite the higher copy number, indicating silencing has been achieved. Additional samples are being processed. A decrease in expression of cpb with a fold change of <0.5 (i.e., that a transgenic individual is expressing the gene at a level less than 50% of average control individuals) is indicative that piRNA cluster formation has occurred, leading to the silencing of the desired host transcript. This mechanism is confirmed with small RNA sequencing to determine if piRNAs mapping to cpb are being expressed.

### Infection of Transgenic and Control Lines

To determine if expression of the receptor blocking peptide or piRNA-mediated down-regulation of host factors required for Plasmodium infection of An. gambiae midguts resulted in lowered transmission capability, malaria infections assays were performed on the transgenic lines and measured the response (Table 2). At 5-7 days post emergence (dpe), female mosquitoes were exposed to P. berghei infected mice (Swiss Webster strain). Females successfully feeding on the provided bloodmeals were then separated from those that did not for subsequent transmission assays. All work involving vertebrate animals was approved by the Johns Hopkins Institutional Animal Care and Use Committee (IACUC).

Table 2 below shows the results from Plasmodium berghei infections on transgenic Anopheles gambiae lines. It is important to note that positively infected midguts identified in transgenic samples were much less obvious than in wild type specimens, and that some of these may have been false positives.

TABLE 2
Results from Plasmodium berghei infections
on transgenic Anopheles gambiae lines.

| | | Intensity of |
|---|---|---|
| Sample | Rate of Infection | Infection |
| Wild type 1 | 0.8 | 20.0 |
| Wild type 2 | 0.8 | 12.6 |
| Wild type 3 | 0.5 | 35.3 |
| Transgenic 1 | 0.4 | 1.2 |
| Transgenic 2 | 0.2 | 1.2 |
| Transgenic 3 | 0.2 | 1.8 |
| Transgenic 4 | 0.4 | 0.8 |
| Transgenic 5 | 0.2 | 1.2 |
| Transgenic 6 | 0.4 | 1.2 |
| Transgenic 7 | 0.7 | 1.3 |

### Transmission Assays

For all infections, a non-transgenic line (Keele strain) of An. gambiae was also infected to provide a control group for cpb expression comparison via qPCR and subsequent RNA sequencing analyses. At 7 dpi the midguts were dissected from transgenic (n=33) and control (n=19) female mosquitoes exposed to Plasmodium to determine the rate (number of infected mosquitoes/number of dissected mosquitoes) and intensity (mean number of oocysts per positive midgut) of infection by oocyst detection.

### Transcriptomic Analyses

qPCR-based analyses are expanded by sequencing total RNA (RNAseq) from transgenic and control females. The sequenced individuals consist of three females each from five different generations of a transgenic line and three females each from three different generations of the wild-type colony population (N=24 samples), and any transgenic females presenting silencing of the host factors are used for small RNA sequencing. Each sample is run in two technical replicates.

Total RNA is processed to separate the transcriptomic from the small RNA fractions. After automated stranded RNA-seq library preparation (with the Roche KAPA RNA HyperPrep Kit), samples are sequenced on the Illumina NovaSeq 6000 platform, targeting at least 30 million reads per sample.

Adapters are trimmed from RNA sequences using the Trimmomatic read trimming software (Bolger, A. M., et al. Bioinformatics, 30 (15), 2114-2120 (2014)). Trimmed reads are mapped to the most recent build of the An. gambiae reference genome (AgamP4) using the STAR RNA sequencing aligner (Dobin, A., et al. Bioinformatics, 29 (1), 15-21 (2013)). Counts of reads mapping to each gene is calculated in R using the Rsubread package (Liao, Y., et al. Nucleic Acids Research, 47 (8) (2019)). Differential expression analysis is performed using a negative binomial general linearized model (GLM) with the edgeR package (Chen, Y., et al. Bioconductor User's Guide (2014); Liao, Y., et al. Nucleic Acids Research, 47 (8) (2019)). Genes that have differential expression by transgenic and infection status are identified, which will inform the overall transcriptomic response to the TE integrations and how TE integrations interfere with the normal molecular response to infection. Genes that are significantly differentially expressed (i.e., those with an absolute value of log-fold change >2 or <0.5 and FDR<0.05) are analyzed with TopGo in R to identify functional categories, such as Gene Ontology (GO) biological processes and molecular functions, that are overrepresented among differentially expressed genes.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A polynucleotide for regulating expression of a target gene, comprising a nucleotide sequence encoding one or more piRNAs or precursors thereof, wherein the one or more piRNAs binds to a Piwi or Aubergine protein and forms a RNP complex (piRC) with the Piwi or Aubergine proteins, and wherein the one or more piRNAs comprise a piRNA nucleotide sequence that hybridizes to at least a portion of a mRNA transcript of the target gene and thus causes downregulation of transcription of the target gene.

2. The polynucleotide of claim 1, wherein the piRNA is about 20 to about 50 nucleotides in length.

3. The polynucleotide of claim 1, wherein the piRNAs are of the same or different sequences.

4. The polynucleotide of claim 1, wherein the piRNA comprises no more than 1 in 5 base pairs of nucleotide mismatches with respect to the mRNA transcript of the target gene.

5. The polynucleotide of claim 1, wherein the piRNA has at least 90% sequence identity with the portion of the mRNA transcript of the target gene to which the piRNA hybridizes.

6. The polynucleotide of claim 1, wherein the piRNA comprises a nucleotide sequence having at least 75% sequence identity with the nucleotide sequence selected from SEQ ID NOs: 1 to 9 or comprises the nucleotide sequence selected from SEQ ID NOs: 1 to 9.

7. The polynucleotide of claim 1, wherein the target gene comprises an insect-specific gene.

8. The polynucleotide of claim 1, wherein the target gene comprises an Anopheles gambiae-specific gene.

9. The polynucleotide of claim 8, wherein the target gene comprises Anopheles gambiae carboxypeptidase B1 (AgCPB-1).

10. The polynucleotide of claim 9, wherein the target gene comprises a nucleotide sequence having at least 75% sequence identity with the nucleotide sequence of SEQ ID NO: 10 or comprises the nucleotide sequence of SEQ ID NO: 10.

11. The polynucleotide of claim 1, comprising a nucleotide sequence encoding an Anopheles gambiae carboxypeptidase-B (AgCPB-3) or variant thereof.

12. The polynucleotide of claim 11, wherein the AgCPB-3 comprises a nucleotide sequence having at least 75% sequence identity with the nucleotide sequence of SEQ ID NO: 12 or comprises the nucleotide sequence of SEQ ID NO: 12.

13. The polynucleotide of claim 12, wherein the one or more piRNA is located in an intronic region of the nucleotide sequence encoding AgCPB-3.

14. The polynucleotide of claim 1, comprising one or more nucleotide sequences, each encoding a midgut lumen receptor blocking protein (MBP) or variant thereof.

15. The polynucleotide of claim 14, comprising one, two, three, four, five, or six nucleotide sequences encoding the MBP or variant thereof.

16. The polynucleotide of claim 14, wherein the MBP comprises a nucleotide sequence having at least 75% sequence identity with the nucleotide sequence of SEQ ID NO: 13 or comprises the nucleotide sequence of SEQ ID NO: 13.

17. The polynucleotide of claim 14, wherein the one or more nucleotide sequences are downstream of the nucleotide sequence encoding AgCPB-3 or variant thereof.

18. The polynucleotide of claim 11, comprising an Anopheles gambiae carboxypeptidase (AgCP) promoter operably linked to the nucleotide sequence encoding the AgCPB-3 or variant thereof.

19. The polynucleotide of claim 18, wherein the AgCP promoter comprises a nucleotide sequence having at least 75% sequence identity with the nucleotide sequence of SEQ ID NO: 14 or comprises the nucleotide sequence of SEQ ID NO: 14.

20. The polynucleotide of claim 1, wherein the polynucleotide is configured for transformation into an Anopheles gambiae germline.

21. The polynucleotide of claim 1, wherein the polynucleotide comprises a nucleotide sequence having at least 75% sequence identity with the nucleotide sequence of SEQ ID NO: 15 or comprises the nucleotide sequence of SEQ ID NO: 15.

22. The polynucleotide of claim 1, wherein the piRNA comprises a 5′-uracil residue.

23. A vector, a cell, or a composition comprising the polynucleotide of claim 1.

24.-28. (canceled)

29. A method for regulating the expression of a target gene in a cell, comprising introducing into the cell the polynucleotide of claim 1, the wherein the one or more piRNAs comprise a piRNA nucleotide sequence that hybridizes to at least a portion of a mRNA transcript of the target gene and thus causes down-regulation of transcription of the target gene.

30.-33. (canceled)

34. A method of reducing transmission capability of an insect host, comprising introducing into a cell of the insect host the polynucleotide of claim 1, wherein the one or more piRNAs comprise a piRNA nucleotide sequence that hybridizes to at least a portion of a mRNA transcript of a target gene and thus causes down-regulation of transcription of the target gene, and wherein down-regulation of the transcription of the target gene reduces transmission of a disease or an infection caused by a pathogen.

35.-38. (canceled)


SEQUENCE LISTING (15 sequences)

SEQ ID NO: 1 - DNA, 43 bp, synthetic construct
  source 1..43: mol_type = other DNA
taccgatgtttctagctttacttcgtcgtatgctaagacgagg

SEQ ID NO: 2 - DNA, 51 bp, synthetic construct
  source 1..51: mol_type = other DNA
cggggttttctatcatctaggctcttcctgtggcttgtagctggctagccg

SEQ ID NO: 3 - DNA, 42 bp, synthetic construct
  source 1..42: mol_type = other DNA
tagccgcagcttacaaaaagtccgccgaatcatatacaagtt

SEQ ID NO: 4 - DNA, 44 bp, synthetic construct
  source 1..44: mol_type = other DNA
aataatgggcaaaaccgtctcagccttgaaattctatagtctca

SEQ ID NO: 5 - DNA, 51 bp, synthetic construct
  source 1..51: mol_type = other DNA
catttgacaaacatctggttgccaaatgtgtcacaactttcacaccatctt

SEQ ID NO: 6 - DNA, 32 bp, synthetic construct
  source 1..32: mol_type = other DNA
taagtgttattttgttttgttctacttgttgc

SEQ ID NO: 7 - DNA, 44 bp, synthetic construct
  source 1..44: mol_type = other DNA
gcgtttgaaactccggagaaactgcaacactcagaaccctttgc

SEQ ID NO: 8 - DNA, 41 bp, synthetic construct
  source 1..41: mol_type = other DNA
gggactcacgacgtatactctgttcttgagccttcatctca

SEQ ID NO: 9 - DNA, 46 bp, synthetic construct
  source 1..46: mol_type = other DNA
caatggcacatattaatgctctgtaagccaaggacctgtcgtaatt

SEQ ID NO: 10 - DNA, 1284 bp, Anopheles gambiae
  source 1..1284: mol_type = genomic DNA
atgagcgaagcaatgaagcggctaacattcgtgaccggctgtttgctggccttggcattcgccaaagccggttcttatcatgaattcgagctgtacaacgtgcgcccggaaacggcggaacagctgtcggtgctgctcaagtggcgcaatgggcaggagattgaggtggacttttgggatgcaccgaaggtgggccgtagcgcacgcatcatggtgaccagggaggatcacaagcgggtggaagagttcctggagcagcacgacatcgagtacgatctggtggcggaagatgtgcaggagttgctgaatcgagagcagcggcgaaatgtggagcacggtcggcggctgaggcgtgactcgaattcgcgtgccactgttaatttcgagcacttctggacgctggacgagatctacgagtatctggacgagctggcggtggcgtacaatgggctggtgcgcgtctcggagatcggtcgtacgcatgaggatcgccctatcaaggccatcactatttcgaccatgggtgcagtcgatcagacccgaccgattgtgtttatggatggaggtattcatgccagagaatgggccggcgtgatgtcggtcatgtacatgatccacgagtttgtggaacattcggaccagtacgccgagcagctgtccaacacggactacgtcatcgtgccggttgccaacccggacgggtacgtctacacccacgagcagaaccgtctgtggcgcaagaaccgttcgccgggcaatgtactgtgctacggcgtggacctgaaccgcaacttccccttccagtgggatcgtacgacgagtgagtgtacgaacaactttgccggccatgccgcttcctcagaaaacgaaaccaaagcactgatcggactgatggatcagtataaggccgccattcgcatgtacctggcggtgcacacgtacggcgagatgattctgtggccgtggggttacgatttcctgcacgcaccgaacgaggacgatctgcagcggttgggcgaacgggcacgcgatgcactggtggcggctggcgggcccgagtacgaggtgggcaattcggccgacattctgtacacggcttccggggcgacggacgactacgcgtacagcctgggcgtgccgtactcgtacacgctcgagctgacgggcggtggatcgcaagggttcgatctgcccgcggccgagctggcgcgcgttacctcgcagacgttcgagctgctgaaggtgttcgggcagcatgcgggcacactgtcggtgacttcgtaa

SEQ ID NO: 11 - DNA, 1269 bp, Anopheles gambiae
  source 1..1269: mol_type = genomic DNA
atgcggtttccagtgctggtggcggttttgtgcgcggtaattggaggtaccgctggccgccaatcgtacagtggctacaagctctactctgtggagcagaacaaccagcagcaggtggacttcctgcgcgagctgcaacagtccgcccaggatcttgacttctggcagcttgatcgtttggttggttccgaggcacgggtgctggtgcctccggcacaattggactacttccgccagctgctgaaggcccagaagttgcgccaccgggaactgattcacgatttcgaacgtgtgcaagatgatcatctgtacggtaccaaacgggtgactccttcgcagggtcttcccctcaacaagtacctgcggtacaacgaaatgatcgactacatcaacacactcgcgaagaagtactccagctttgtaaccgttgccgaaattggcaaatcctacgaaggacgcccaatcccggccgtcaccatccagtccccgtcgctctacaaatcgcacccgaacagctccaaaccggtcgtattcgtcgatgctggcattcatgcgcgtgaatgggccgcacctgccatggcgatgtatctgatcagcgagctggtggagaatgccgcccagcatcaggatctactcgccgggcttacctggacgatcgtgccaattgccaatccggacgggtacgagtacagtcacgagcgggaacgcctgtggcgcaaaacgcgccgtccggccggacgtaactgtgtcggcgtcgatggcaaccgcaactacgacttccactgggcggaggtcggtgcctcgaatcagccctgctccgacacataccacggggagcagtcgttctctgagccggagacgcgcgccatccgggacgagctgctgaggttgaagggacgctgcaagttctacctgtcgctgcacacgtacggacagtacctgctgtacccgtggggatggacgtcggagctgccggaggactggcagaaggtcgatgccgttgcgcgggctggagcacgtgcgattgagcaagcgaccgggtcgacctatacggtcggcagctccaccaatgtgctgtatgcggccgctggaggcagtgatgattacgcgtatgctgtcgccgatgtgccgatttcgatgacgatggaactgccgggcggtggttcgcagggcttcaatccaccgccgacgcgcatcgaagagatcgtgaaggaaacgttcgtgggcgtgcgggcgatggcgttggaggtggcgcgaaactacagctga

SEQ ID NO: 12 - DNA, 1248 bp, Anopheles gambiae
  source 1..1248: mol_type = genomic DNA
atgaagtactgtgctgccgtggttgtgctcctagcgctggccgtgtttggccagagcgccttcgccgaacaggtgtcctaccgagactacaaagtctacagcatccgcgtggacacggtggagaagcacaacgtgctgaagcgctggcaggatgtgcgcggggtcgatttctgggaccgtgccggctaccgtgtgatgatccatccgagcctgcaggaagcgttcgagcggttcctgaacctgaacgcgttcagctacgagcgcatcattgaggatgtggaagccacgatcgaggcagagcgcaagtacgatcaggagtaccgccgtcgcaaggctgcatccggtcgggctacggtcgatttcgagcacttctggacgaatgcggaggttaatgcgtacctggacgagcttgcccagacgtacccgaatctggtgcgcgtcgccacgattggcaccactcacgagggccgtccgatcaagtcgatcacgatctcgaccaacaatggtgtggctggatcgaagccggttgtcttcatcgacggtggtatccatgcacgtgaatgggccggtgtaatgtcggtgctctacttgatccacgagctggtggaacattcgagcagctacgccgacatgctgaacaaggactgggtgatcattcccgtcgccaacccggatgggtacgagttctcgcacaccgacaaccgcatgtggcgcaagaaccgcttcccggccacgatcctctgtaccggtattgatctgaaccgtaactgggactatctgtgggtgtttagcagcaatgcctgctccgactcgtacgccggaacgaccgccttctccgagctggaaacgcaagccctcgatcgggtgctgaagcagtacggatcgaacattgcggtctatctggcggtgcacacgtacggcgatatgatcctgtacccgtacggacactcgtggccgttcgtgccggtcgctaaccaggccgcccacattgcgctgggtgagcaggcgcgcgatgcaatcacggccgtcggtggcccccggtacgtggtgggcaacagtgcggaaattctgtacacggccaacggttgcagcgatgattatgtggccggtgtgatcggcgcgcggtacgcgtacacgctcgagctgacgggcggtggccgcaatgggttcgatctgcctgccacggaaattatggctgtggcgacccaaaccttccagatctatcgcaccatggcgaacaatgcgtaa

SEQ ID NO: 13 - DNA, 36 bp, synthetic construct
  source 1..36: mol_type = other DNA
ccgtgccagcgcgcgatcttccagtcgatctgcaac

SEQ ID NO: 14 - DNA, 3408 bp, Anopheles gambiae
  source 1..3408: mol_type = genomic DNA
ccgaactcaaaactgaaaacgctaacacctattctgaaagagggtatcctacgtgtgggtggtcgtttgcgaaacgcgcctgtttcgtatgaacgcaaacatccgataattttagcgttttctcacccattgaccctactgattgcgcgttcttatcatcgacaatatctgcatgcgggacaacaagagctcatatctagcctgcgtgagagattctggccccttcgcgtacgcaacctggcacgaaaggtcgtatatgagtgtgtaagttgtttccgatccaaaccaacaacggcagagcaaatcatgggagatttgcccagcgaacgcgtaaacccagttctcccattctacaacactggtgtggatctgtgtggtccattattctatagacaaaccaacaagaaggctgctccaatcaaatgctatgttgctgttttcgtctgtcttgtgatcaaggcagtacatgtggagcttattgccgatttgtctactccagccttcatttctacattgaagcgtttcatagctcgtcgcggtaaaccatccgtaatccagtgcgacaacgccaaaaatttccggggagctgatcgagcgctcaaggagatgtatgagctgttccagaaacaacaacatcaggatgctgttacaacttactgcggaacggaagggatcaccttcaacttcatccctccgcggtcaccccatttaaaacggtgggatctgggaggccgcagtgaagtcgctgaaacggcatctcaaggctacgataggatccagtatcttgcggcgagacgacctggaaactatcttagttcaagtggaggcttgtttgaactcgcgaccgttaactgcactttccaacgatccagaggacctggaaattctgacaccgggtcattttttgattcaacgggcgcttacatcggtccctgagccatcttatgctgagattccaggcaaccgcctggaccgctatcaacaattgcaggagtatgttcggcggatttggaagcgatggaatcgagactacttgtctggtttgcatccgcgtacccggtggacttcaaggcgagacaacgttcgggagggcactatggtcctgctgaaggaggacaaccttccccctctcaaatggcgcttcggccgagtgctgaagatttatcctggtgatgacggcttggttcgagtggttgatgtgaaaacgaaggatggaatttacagaagagccatcaccaagatctgtatactgcccggacagaaggaagaaagagaggtttcgtaataagggttgaaagctacctttcaacggggggcggctatgttgagtaatgaatttgtacggtataaacgtatgaccttctgtcacttgaatttgttgcttgggaattgtattgaggggacgaatgacggaaacaaggaataaagaaagagtctagcgcaagcgttcaatcagtacagacgttcctctctctaccagttgagaaattcactaactaaaacagtaacaactgaaccgttttgatatcccatctgtttgtataggtattgattgtgaacagcaatgtgtacgataggaatcgtgatatgaaaatgctgatcctaaaacagcgctgcctcatcgaaagctaggaatcgtttgtgaaatgtaataaaacgtatttaaccttcgttgtaactattttaattgttatcgaattgttttgctgaatcaaaacgttctaagatacattaaaataaacgtatgcatggtacgatttttacttaataacgctttagcaaaataaggtttgagtctacaaaacttgtttttactgaatctattattattgcttattcacaatactgattgatggctttgatagttctagaaaaatattcaattttcgaaaaccgcatagaagcagaatggcgcaatatatttttgctcttgggattgggggctgtaagtttgacacctctggcttcggtcggatggcggatggtcgtgaaggttcggtttcttaagacactattttccttattcttgtgaaactagtcttatgaattgtcactttttatctttttctctctctcattctctctctctatttctttctctttctctctctctctttctctctctctctttctctatctctctctttctctatctcgttctatctctctatttctcaccctatcgttgaaaaaatctgttttagtgatgattgttaaattaaatataaaacttgcattaaatatcttcaaacatttgaaagatagcatttagagctacatttctagcgattagttaattagctaattattaaattagtaagacgtgcacgatctaatgcaaatcaacccagttaacagtggtaagcatgttatcataaagaggagtttatgcatttttattacgcacttcttcattctacttctctgttctatcaaatgaagaaaagttcattagaatgatcacctcactgtgctgctatcagtatcacaccgaaactctcgacccagttcgtagaatttgcttaccatcatttgcctcatttttttgcgcctcacatcctgaacccgtcgtgagcaagtcgcgagccagaccctgtctgttatttccattctccaaacacccggtgtggcccataagttgtacgttttcacacatgtacctgttatcgatgacaagcgccatccgtatcagtgtgctgcgatctacacctgcatcttggttcggttatcgccgagcatagaatcagctgttgatagggcagtgcgataagcgtacttaagaaattggagcaattatggagtgttggttaaggcaggtgggggaaaagtaaataatcacgatggggtacggaatgggtttgaaaacaaaagccaatcaagcacattagccaaagcatatctaaagggggattaatgagagtttgtgttttgtttaagcctattgcgctttcgtaaatgtttcgtacgacaatctcatgtgctaattctttgaatttccatgcatgaaccaaggtttccccttctcagacagtatgcatatctttagaaagatcatatcgctctcgaactagataagatcaagccattcacgataggcgtgtaccagtcaatgtttgtcaccgtcactcaataaatactgaggacctcaatactccagggtttggtttactgttcatccaattccagtaatcgattccggttagtagattaagcaaagctgtccacggacaacacgcagtctgtgtgttctgcagacattcgttatctaatcgtacgtgattcaatcaaacgcaatatcgcaatgacaagagtttcgcgtgcgttcaaagtcgtgaaatcattaagccgcacatcattacacactgggggttggaaattttggcttttattcgcgcgtcatagccgtcc

SEQ ID NO: 15 - DNA, 6210 bp, synthetic construct
  source 1..6210: mol_type = other DNA
ccgaactcaaaactgaaaacgctaacacctattctgaaagagggtatcctacgtgtgggtggtcgtttgcgaaacgcgcctgtttcgtatgaacgcaaacatccgataattttagcgttttctcacccattgaccctactgattgcgcgttcttatcatcgacaatatctgcatgcgggacaacaagagctcatatctagcctgcgtgagagattctggccccttcgcgtacgcaacctggcacgaaaggtcgtatatgagtgtgtaagttgtttccgatccaaaccaacaacggcagagcaaatcatgggagatttgcccagcgaacgcgtaaacccagttctcccattctacaacactggtgtggatctgtgtggtccattattctatagacaaaccaacaagaaggctgctccaatcaaatgctatgttgctgttttcgtctgtcttgtgatcaaggcagtacatgtggagcttattgccgatttgtctactccagccttcatttctacattgaagcgtttcatagctcgtcgcggtaaaccatccgtaatccagtgcgacaacgccaaaaatttccggggagctgatcgagcgctcaaggagatgtatgagctgttccagaaacaacaacatcaggatgctgttacaacttactgcggaacggaagggatcaccttcaacttcatccctccgcggtcaccccatttaaaacggtgggatctgggaggccgcagtgaagtcgctgaaacggcatctcaaggctacgataggatccagtatcttgcggcgagacgacctggaaactatcttagttcaagtggaggcttgtttgaactcgcgaccgttaactgcactttccaacgatccagaggacctggaaattctgacaccgggtcattttttgattcaacgggcgcttacatcggtccctgagccatcttatgctgagattccaggcaaccgcctggaccgctatcaacaattgcaggagtatgttcggcggatttggaagcgatggaatcgagactacttgtctggtttgcatccgcgtacccggtggacttcaaggcgagacaacgttcgggagggcactatggtcctgctgaaggaggacaaccttccccctctcaaatggcgcttcggccgagtgctgaagatttatcctggtgatgacggcttggttcgagtggttgatgtgaaaacgaaggatggaatttacagaagagccatcaccaagatctgtatactgcccggacagaaggaagaaagagaggtttcgtaataagggttgaaagctacctttcaacggggggcggctatgttgagtaatgaatttgtacggtataaacgtatgaccttctgtcacttgaatttgttgcttgggaattgtattgaggggacgaatgacggaaacaaggaataaagaaagagtctagcgcaagcgttcaatcagtacagacgttcctctctctaccagttgagaaattcactaactaaaacagtaacaactgaaccgttttgatatcccatctgtttgtataggtattgattgtgaacagcaatgtgtacgataggaatcgtgatatgaaaatgctgatcctaaaacagcgctgcctcatcgaaagctaggaatcgtttgtgaaatgtaataaaacgtatttaaccttcgttgtaactattttaattgttatcgaattgttttgctgaatcaaaacgttctaagatacattaaaataaacgtatgcatggtacgatttttacttaataacgctttagcaaaataaggtttgagtctacaaaacttgtttttactgaatctattattattgcttattcacaatactgattgatggctttgatagttctagaaaaatattcaattttcgaaaaccgcatagaagcagaatggcgcaatatatttttgctcttgggattgggggctgtaagtttgacacctctggcttcggtcggatggcggatggtcgtgaaggttcggtttcttaagacactattttccttattcttgtgaaactagtcttatgaattgtcactttttatctttttctctctctcattctctctctctatttctttctctttctctctctctctttctctctctctctttctctatctctctctttctctatctcgttctatctctctatttctcaccctatcgttgaaaaaatctgttttagtgatgattgttaaattaaatataaaacttgcattaaatatcttcaaacatttgaaagatagcatttagagctacatttctagcgattagttaattagctaattattaaattagtaagacgtgcacgatctaatgcaaatcaacccagttaacagtggtaagcatgttatcataaagaggagtttatgcatttttattacgcacttcttcattctacttctctgttctatcaaatgaagaaaagttcattagaatgatcacctcactgtgctgctatcagtatcacaccgaaactctcgacccagttcgtagaatttgcttaccatcatttgcctcatttttttgcgcctcacatcctgaacccgtcgtgagcaagtcgcgagccagaccctgtctgttatttccattctccaaacacccggtgtggcccataagttgtacgttttcacacatgtacctgttatcgatgacaagcgccatccgtatcagtgtgctgcgatctacacctgcatcttggttcggttatcgccgagcatagaatcagctgttgatagggcagtgcgataagcgtacttaagaaattggagcaattatggagtgttggttaaggcaggtgggggaaaagtaaataatcacgatggggtacggaatgggtttgaaaacaaaagccaatcaagcacattagccaaagcatatctaaagggggattaatgagagtttgtgttttgtttaagcctattgcgctttcgtaaatgtttcgtacgacaatctcatgtgctaattctttgaatttccatgcatgaaccaaggtttccccttctcagacagtatgcatatctttagaaagatcatatcgctctcgaactagataagatcaagccattcacgataggcgtgtaccagtcaatgtttgtcaccgtcactcaataaatactgaggacctcaatactccagggtttggtttactgttcatccaattccagtaatcgattccggttagtagattaagcaaagctgtccacggacaacacgcagtctgtgtgttctgcagacattcgttatctaatcgtacgtgattcaatcaaacgcaatatcgcaatgacaagagtttcgcgtgcgttcaaagtcgtgaaatcattaagccgcacatcattacacactgggggttggaaattttggcttttattcgcgcgtcatagccgtcccggtttattttttatgggatgagattaactgccttcagctcgatagcaggaagaaaggtataaattgtaccgccactggttagtcgccttcatctctgtacgggtcactgttgcaggattagctaaaaatgaagtactgtgctgccgtggttgtgctcctagcgctggccgtgtttggccagagcgccttcgccgaacaggtgtcctaccgagagtgagtgttctttcaccagacaaagtaccgatgtttctagctttacttcgtcgtatgctaagacgaggcggggttttctatcatctaggctcttcctgtggcttgtagctggctagccgcagcttacaaaaagtccgccgaatcatatacaagttaataatgggcaaaaccgtctcagccttgaaattctatagtctcacatttgacaaacatctggttgccaaatgtgtcacaactttcacaccatctttaagtgttattttgttttgttctacttgttgcgcgtttgaaactccggagaaactgcaacactcagaaccctttgcgggactcacgacgtatactctgttcttgagccttcatctcacaatggcacatattaatgctctgtaagccaaggacctgtcgtaatttgtgtccgaaaagagccgaaaattgatgcgtgaaaaatcaatccaattccagctacaaagtctacagcatccgcgtggacacggtggagaagcacaacgtgctgaagcgctggcaggatgtgcgcggggtcgatttctgggaccgtgccggctaccgtgtgatgatccatccgagcctgcaggaagcgttcgagcggttcctgaacctgaacgcgttcagctacgagcgcatcattgaggatgtggaagccacgatcgaggcagagcgcaagtacgatcaggagtaccgccgtcgcaaggctgcatccggtcgggctacggtcgatttcgagcacttctggacgaatgcggaggttaatgcgtacctggacgagcttgcccagacgtacccgaatctggtgcgcgtcgccacgattggcaccactcacgagggccgtccgatcaagtcgatcacgatctcgaccaacaatggtgtggctggatcgaagccggttgtcttcatcgacggtggtatccatgcacgtgaatgggccggtgtaatgtcggtgctctacttgatccacgagctggtggaacattcgagcagctacgccgacatgctgaacaaggactgggtgatcattcccgtcgccaacccggatgggtacgagttctcgcacaccgacaaccgcatgtggcgcaagaaccgcttcccggccacgatcctctgtaccggtattgatctgaaccgtaactgggactatctgtgggtgtttagcagcaatgcctgctccgactcgtacgccggaacgaccgccttctccgagctggaaacgcaagccctcgatcgggtgctgaagcagtacggatcgaacattgcggtctatctggcggtgcacacgtacggcgatatgatcctgtacccgtacggacactcgtggccgttcgtgccggtcgctaaccaggccgcccacattgcgctgggtgagcaggcgcgcgatgcaatcacggccgtcggtggcccccggtacgtggtgggcaacagtgcggaaattctgtacacggccaacggttgcagcgatgattatgtggccggtgtgatcggcgcgcggtacgcgtacacgctcgagctgacgggcggtggccgcaatgggttcgatctgcctgccacggaaattatggctgtggcgacccaaaccttccagatctatcgcaccatggcgaacaatgcgtaagcgttggggacaattttaatataattaaaggtttttttttacaaaaaaaaactgtaactgcatgtttgagatgttcttatttcttggaagacggttacatctcgatcaagagaaagagagagagagaaagagatgacatgcactgctacagagctcattagcgttatggggctcgccgggcccgtgccagcgcgcgatcttccagtcgatctgcaacggctcgccgggcccgtgccagcgcgcgatcttccagtcgatctgcaacggctcgccgggcccgtgccagcgcgcgatcttccagtcgatctgcaacggctcgccgggcccgtgccagcgcgcgatcttccagtcgatctgcaactaattgtaacagttagttatctattatttaatatactgctatcgatacaatcgggcgcacgcgtgcgccacccagttcctgatactatcacgacgcctcccttccaattagtgaaattggtgatcaggttttgaaatcaacgtaagatgttgtctgtgtgcaagaaccgcatccagtttatatggctgtgggttgcgctggaaccgtgtcagtcagactcctgcaacttccgctgccacaatgaagctactcgcggtggtggtgtttctgttcaccgtcggcaccgtttgccgggcggagcagcaaaagttcgaaaagtaggtatgcggtacaatagaaggattgttctatttcatcaattcacaatccctcctaacagctacaagctctactcactgtacgtcgaggatgacgaacaggcggaagagctacacaactggtacgccgacgggaagctggacttctggagctacggcacactgaacgacagcgtcaccgtgatggtgcagcccgatctgcaggaatactttgaggctatgctggacgatgtgcagctcgaatcggagctggtagaggagaacgtacagtcactgctcgagc